(19) United States
(12) Patent Application Publication
(10) Pub. No.: US 2022/0315540 A1
(43) Pub. Date: Oct. 6, 2022

(54) LIGHT-EMITTING DEVICE, LIGHT-EMITTING SUBSTRATE AND LIGHT-EMITTING APPARATUS

(71) Applicant: BOE TECHNOLOGY GROUP CO., LTD., Beijing (CN)

(72) Inventors: Bingyan LIANG, Beijing (CN); Rongrong GAO, Beijing (CN)

(73) Assignee: BOE TECHNOLOGY GROUP CO., LTD., Beijing (CN)

(21) Appl. No.: 17/615,498

(22) PCT Filed: Oct. 22, 2020
(86) PCT No.: PCT/CN2020/122959

§ 371 Date: Nov. 30, 2021

(51) Int. Cl. C07D 251/24; C07D 471/04; C07D 277/64; C07D 241/12; H01L 51/00

(52) U.S. Cl. CPC C07D 251/24; C07D 471/04; C07D 277/64; C07D 241/12; H01L 51/0072; H01L 51/0067; H01L 51/0056; H01L 51/5072

(57) ABSTRACT

A light-emitting device includes a hole blocking layer including one or more of compounds shown in following formula (1)a and formula (1)b.

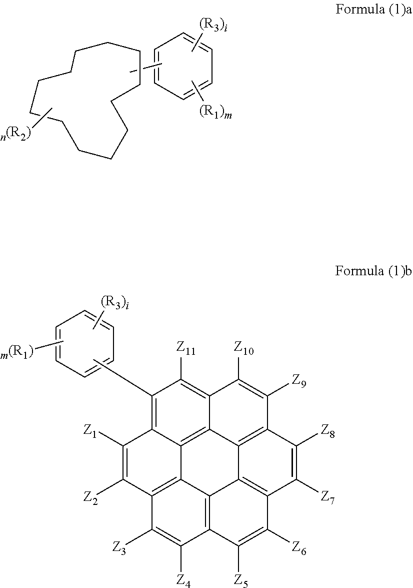

Z₁ to Z₁₁ are selected from H or R. R, R₁ and R₂ are each selected independently from deuterium, halogen, cyano, nitryl, amino, C₁-C₄₀ alkyl, C₂-C₄₀ alkenyl, C₂-C₄₀ alkynyl, C₃-C₄₀ cycloalkyl, C₃-C₄₀ heterocycloalkyl, C₆-C₆₀ aryl, C₅-C₆₀ heteroaryl, C₁-C₄₀ alkoxy, C₆-C₆₀ aryloxy, C₃-C₄₀ alkylsilyl, C₆-C₆₀ arylsilyl, C₁-C₄₀ alkylboron, C₆-C₆₀ arylboron, C₆-C₆₀ arylphosphinylene, C₆-C₆₀ monoaryl or diaryl phosphino and C₆-C₆₀ arylamino. R₃ is selected from heterocyclyl and fused heterocyclyl.

### CROSS-REFERENCE TO RELATED APPLICATION

This application is a national phase entry under 35 USC 371 of International Patent Application No. PCT/CN2020/122959 filed on Oct. 22, 2020, which is incorporated herein by reference in its entirety.

### TECHNICAL FIELD

The present disclosure relates to the field of illumination and display technologies, and in particular, to a light-emitting device, a light-emitting substrate and a light-emitting apparatus.

### BACKGROUND

An organic light-emitting diode (OLED), which is known as a next-generation “star” display technology, has characteristics of self-luminescence, wide visible angle, fast response time, high luminous efficiency, low operating voltage, small substrate thickness, capability of constituting a large size and flexible substrate, simple manufacturing process and the like.

### SUMMARY

In an aspect, a light-emitting device is provided, including: a first electrode and a second electrode that are stacked, a light-emitting layer between the first electrode and the second electrode, an electron transport layer between the first electrode and the light-emitting layer, and a hole blocking layer between the light-emitting layer and the electron transport layer. A material of the hole blocking layer includes one or more of compounds, containing coronene or cyclododecane, shown in the following formula (1)a and formula (1)b.

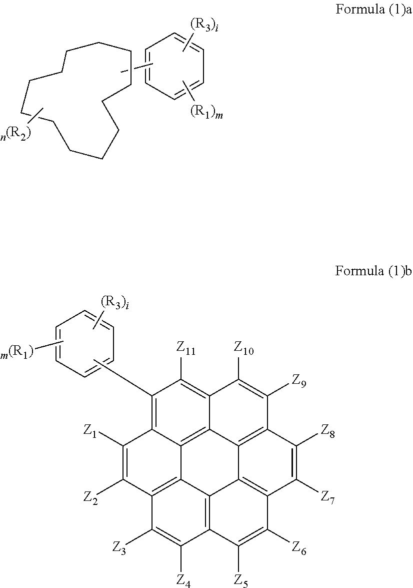

m is an integer from 0 to 2, n is an integer from 0 to 5, and i is an integer from 0 to 3. In the formula (1)a, at least one of m, n, and i is not 0, and in the formula (1)b, at least one of m and i is not 0.

Z₁ to Z₁₁ are the same or different, and are each selected independently from any one of hydrogen (H) and a substituent R.

The substituent R, a substituent R₁ and a substituent R₂ are the same or different, and are each selected independently from any one of deuterium, halogen, cyano, nitryl, amido, C₁-C₄₀ alkyl, C₂-C₄₀ alkenyl, C₂-C₄₀ alkynyl, C₃-C₄₀ cycloalkyl, C₃-C₄₀ heterocycloalkyl, C₆-C₆₀ aryl, C₅-C₆₀ heteroaryl, C₁-C₄₀ alkoxy, C₆-C₆₀ aryloxy, C₃-C₄₀ alkylsilyl, C₆-C₆₀ arylsilyl, C₁-C₄₀ alkylboron, C₆-C₆₀ arylboron, C₆-C₆₀ arylphosphinylene, C₆-C₆₀ monoaryl or diaryl phosphino and C₆-C₆₀ arylamino, or are each independently combined with an adjacent group to form a condensed ring.

R₃ is selected from any one of substituted or unsubstituted heterocyclyl and fused heterocyclyl.

In some embodiments, i is not 0, and R₃ is selected from any one of the following formulas (3)a, (3)b, (3)c, (3)d and (3)e.

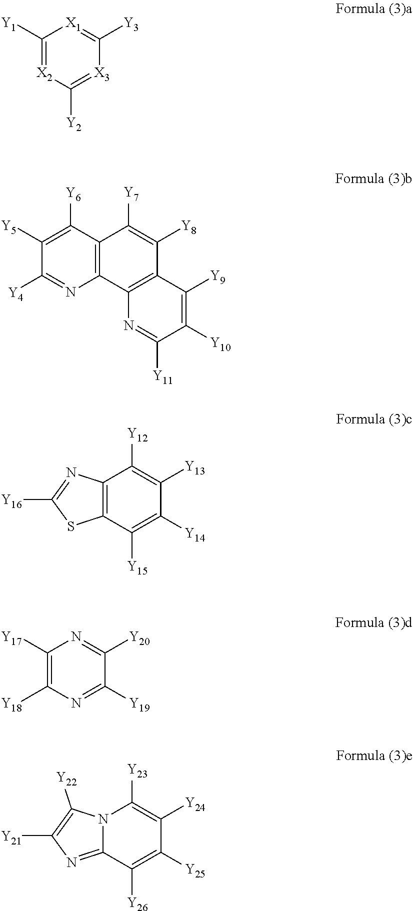

In the formula (3)a, X₁ to X₃ are each C(Y) or N, and at least two of X₁ to X₃ are N. One of Y and Y₁ to Y₃ is combined with a dotted line in the formula (1)a or a dotted line in the formula (1)b, and the others are the same or different, and are each selected independently from any one of the hydrogen and the substituent R.

In the formula (3)b, one of Y₄ to Y₁₁ is combined with the dotted line in the formula (1)a or the dotted line in the formula (1)b, and the others are the same or different, and are each selected independently from any one of the hydrogen and the substituent R.

In the formula (3)c, one of Y₁₂ to Y₁₆ is combined with the dotted line in the formula (1)a or the dotted line in the formula (1)b, and the others are the same or different, and are each selected independently from any one of the hydrogen and the substituent R.

In the formula (3)d, one of Y₁₇ to Y₂₀ is combined with the dotted line in the formula (1)a or the dotted line in the formula (1)b, and the others are the same or different, and are each selected independently from any one of the hydrogen and the substituent R.

In the formula (3)e, one of Y₂₁ to Y₂₆ is combined with the dotted line in the formula (1)a or the dotted line in the formula (1)b, and the others are the same or different, and are each selected independently from any one of the hydrogen and the substituent R.

In some embodiments, in the formula (1)a, at least m of m and n is not 0, and the substituent R₁ is selected from the structure shown in the following formula (2).

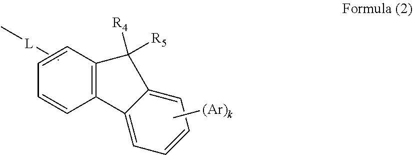

In the formula (2), R₄ and R₅ are the same or different, and are each selected independently from any one of deuterium, halogen, cyano, nitryl, C₁-C₄₀ alkyl, C₂-C₄₀ alkenyl, C₂-C₄₀ alkynyl, C₃-C₄₀ cycloalkyl, C₃-C₄₀ heterocycloalkyl, C₆-C₆₀ aryl, C₅-C₆₀ heteroaryl, C₁-C₄₀ alkoxy, C₆-C₆₀ aryloxy, C₃-C₄₀ alkylsilyl, C₆-C₆₀ arylsilyl, C₁-C₄₀ alkylboron, C₆-C₆₀ arylboron, C₆-C₆₀ arylphosphinylene, C₆-C₆₀ monoaryl or diaryl phosphino and C₆-C₆₀ arylamino, or are each independently combined with an adjacent group to form a condensed ring.

k is an integer from 0 to 2, Ar is selected from any one of C₁-C₁₀ alkyl, C₃-C₁₀ cycloalkyl, and C₆-C₃₀ aryl, and L is selected from any one of a single bond, substituted or unsubstituted divalentaryl, and substituted or unsubstituted divalentheteroaryl.

In some embodiments, in the formula (1)a, m and n are both not 0, and the substituent R₂ and the substituent R₁ are the same or different.

In some embodiments, n is greater than 1, and substituents R₂ are the same or different.

In some embodiments, the material of the hole blocking layer is selected from one or more of the structures shown in the following formulas:

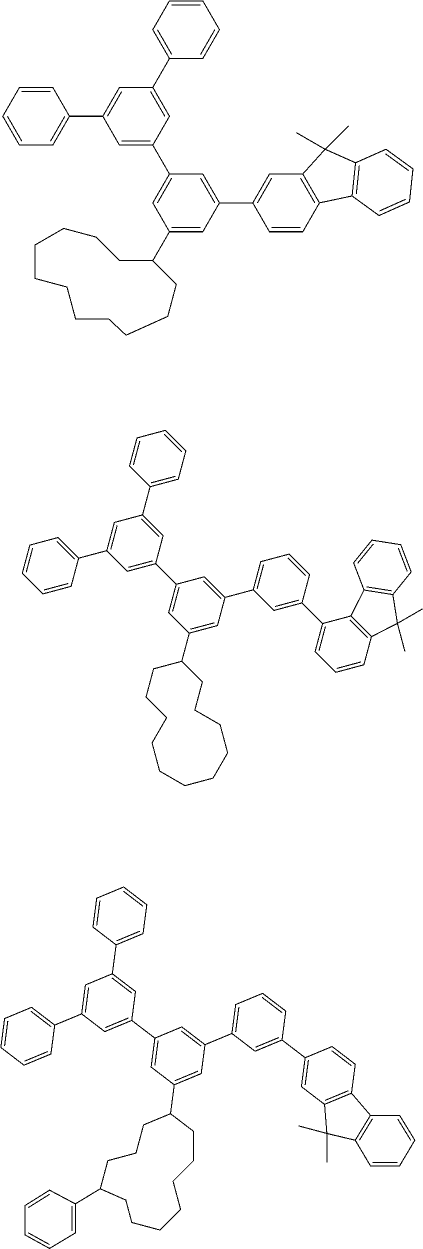

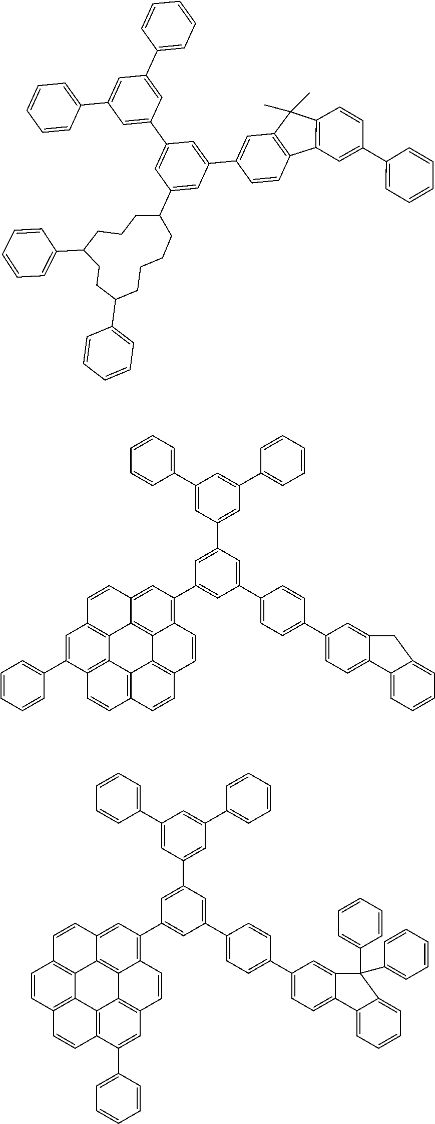

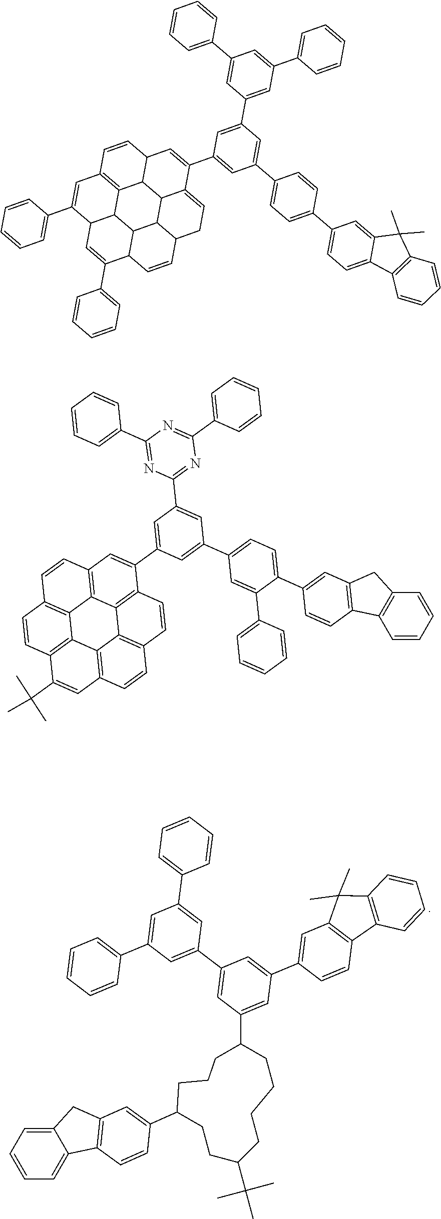

In some embodiments, highest occupied molecular orbital (HOMO) energy levels of the compounds containing the coronene or the cyclododecane are less than −5.6 eV.

In some embodiments, a HOMO energy level of a compound in the compounds containing the coronene or the cyclododecane and a lowest unoccupied molecular orbital (LUMO) energy level of the compound containing the coronene or the cyclododecane meet the following formula:

|E_{HOMO}−E_{LUMO}|≥3.2 eV.

In some embodiments, lowest singlet energy of the compounds containing the coronene or the cyclododecane is greater than 3.1 eV.

In some embodiments, a difference between lowest singlet energy of a compound in the compounds containing the coronene or the cyclododecane and lowest triplet energy of the compound containing the coronene or the cyclododecane is greater than 0.51 eV.

In some embodiments, glass transition temperatures of the compounds containing the coronene or the cyclododecane are within a range of 136° C. to 153° C., inclusive.

In some embodiments, an energy level difference between a HOMO energy level of the light-emitting layer and a HOMO energy level of the hole blocking layer is greater than 0.2 eV.

In some embodiments, an absolute value of an energy level difference between a LUMO energy level of the light-emitting layer and a LUMO energy level of the hole blocking layer is less than 0.3 eV.

In some embodiments, an absolute value of an energy level difference between a LUMO energy level of the hole blocking layer and a LUMO energy level of the electron transport layer is less than 0.3 eV.

In some embodiments, an absolute value of an energy level difference between a HOMO energy level of the hole blocking layer and a HOMO energy level of the electron transport layer is greater than 0.2 eV.

In some embodiments, a material of the electron transport layer includes one or more of the compounds, containing the coronene or the cyclododecane, shown in the formula (1)a and the formula (1)b.

In some embodiments, a difference between energy of the hole blocking layer in a lowest singlet excited state and energy of the light-emitting layer in the lowest singlet excited state is greater than 0.2 eV, and a difference between energy of the hole blocking layer in a lowest triplet excited state and energy of the light-emitting layer in the lowest triplet excited state is greater than 0.2 eV.

In some embodiments, a material of the light-emitting layer includes a host material and a guest material. The host material is selected from any one of anthracene, benzanthracene, benzophenanthrene and/or pyrene compounds and derivatives of these compounds, and atropisomers of these compounds and atropisomers of derivatives of these compounds. The guest material is selected from arylamino type compounds.

In another aspect, a light-emitting substrate is provided, including the light-emitting device described above.

In yet another aspect, a light-emitting apparatus is provided, including the light-emitting substrate described above.

### BRIEF DESCRIPTION OF THE DRAWINGS

In order to describe technical solutions in the present disclosure more clearly, accompanying drawings to be used in some embodiments of the present disclosure will be introduced briefly below. However, the accompanying drawings to be described below are merely accompanying drawings of some embodiments of the present disclosure, and a person of ordinary skill in the art may obtain other drawings according to these drawings. In addition, the accompanying drawings to be described below may be regarded as schematic diagrams, but are not limitations on actual sizes of products, actual processes of methods and actual timings of signals to which the embodiments of the present disclosure relate.

FIG. 1 is a sectional structural view of a light-emitting device, in accordance with some embodiments;

FIG. 2 is a sectional structural view of a light-emitting device, in accordance with some other embodiments; and

FIG. 3 is a sectional structural view of a light-emitting substrate, in accordance with some embodiments.

### DETAILED DESCRIPTION

Technical solutions in some embodiments of the present disclosure will be described clearly and completely with reference to the accompanying drawings below. Obviously, the described embodiments are merely some but not all embodiments of the present disclosure. All other embodiments obtained by a person of ordinary skill in the art based on the embodiments of the present disclosure shall be included in the protection scope of the present disclosure.

Unless the context requires otherwise, throughout the description and the claims, the term “comprise” and other forms thereof such as the third-person singular form “comprises” and the present participle form “comprising” are construed as an open and inclusive meaning, i.e., “including, but not limited to.” In the description of the specification, the terms such as “one embodiment”, “some embodiments”, “exemplary embodiments”, “example”, “specific example” or “some examples” are intended to indicate that specific features, structures, materials or characteristics related to the embodiment(s) or example(s) are included in at least one embodiment or example of the present disclosure. Schematic representations of the above terms do not necessarily refer to the same embodiment or example. In addition, the specific features, structures, materials, or characteristics may be included in any one or more embodiments or examples in any suitable manner.

Hereinafter, the terms such as “first” and “second” are only used for descriptive purposes, and are not to be construed as indicating or implying relative importance or implicitly indicating the number of indicated technical features. Thus, features defined as “first” or “second” may explicitly or implicitly include one or more of the features. In the description of the embodiments of the present disclosure, the term “a plurality of” or “the plurality of” means two or more unless otherwise specified.

The phrase “at least one of A, B and C” has a same meaning as the phrase “at least one of A, B or C”, and they both include the following combinations of A, B and C: only A, only B, only C, a combination of A and B, a combination of A and C, a combination of B and C, and a combination of A, B and C.

The phrase “A and/or B” includes the following three combinations: only A, only B, and a combination of A and B.

Use of the phrase “applicable to” or “configured to” is meant an open and inclusive expression, which does not exclude devices that are applicable to or configured to perform additional tasks or steps.

In addition, the use of the phrase “based on” means openness and inclusiveness, since processes, steps, calculations or other actions “based on” one or more of the stated conditions or values may, in practice, be based on additional conditions or values exceeding those stated.

Exemplary embodiments are described herein with reference to cross-sectional views and/or plan views as idealized exemplary drawings. In the accompanying drawings, thickness of layers and regions may be exaggerated for clarity. Therefore, variations in shape with respect to the drawings due to, for example, manufacturing technologies and/or tolerances may be conceivable. Therefore, the exemplary embodiments should not be construed as being limited to the shapes of the regions shown herein, but including shape deviations due to, for example, manufacturing. For example, an etched region shown in a rectangular shape generally has a curved feature. Therefore, the regions shown in the accompanying drawings are schematic in nature, and their shapes are not intended to show actual shapes of the region in a device, and are not intended to limit the scope of the exemplary embodiments.

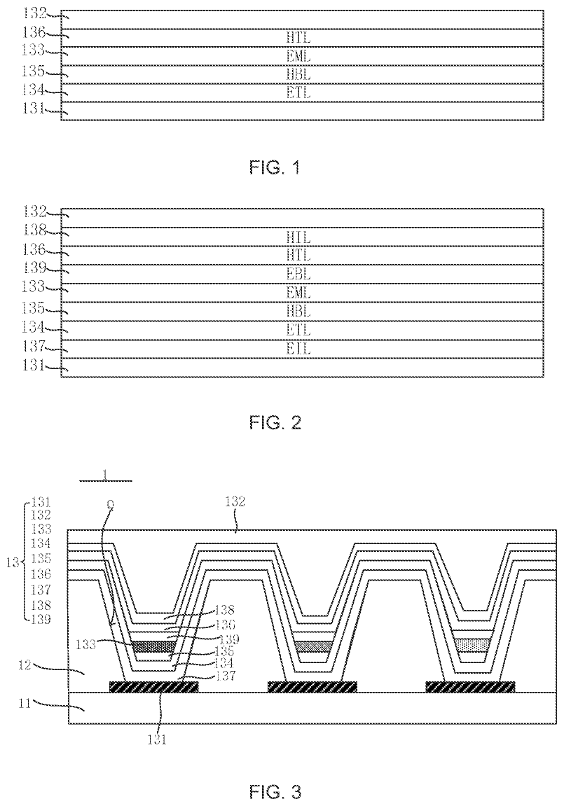

In some embodiments of the present disclosure, a light-emitting device is provided. As shown in FIG. 1, the light-emitting device 13 includes a first electrode (a cathode) 131 and a second electrode (an anode) 132 that are stacked, a light-emitting layer (EML) 133 located between the first electrode 131 and the second electrode 132, an electron transport layer (ETL) 134 located between the first electrode 131 and the light-emitting layer 133, a hole blocking layer (HBL) 135 located between the light-emitting layer 133 and the electron transport layer 134, and a hole transport layer (HTL) 136 located between the second electrode 132 and the light-emitting layer 133. A material of the hole blocking layer 135 includes one or more of compounds, containing coronene or cyclododecane, shown in the following formula (1)a and formula (1)b.

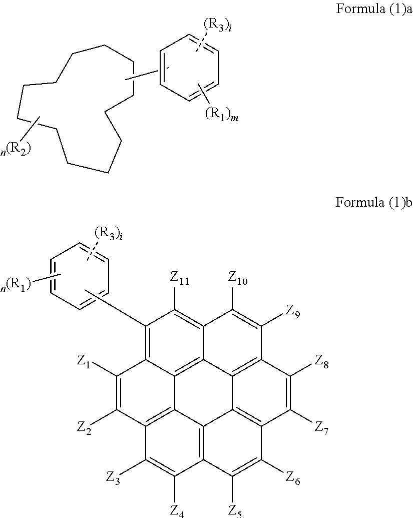

Where m is an integer from 0 to 2, n is an integer from 0 to 5, and i is an integer from 0 to 3. In the formula (1)a, at least one of m, n, and i is not 0, and in the formula (1)b, at least one of m and i is not 0.

That m is an integer from 0 to 2 means that there may be 0 to 2 substituents R₁ on a benzene ring. In a case where there are two substituents R₁, the two substituents R₁ are present on different carbon atoms of the benzene ring. In a case where m is 0, the benzene ring is not substituted by the substituent R₁. In this case, in the formula (1)a, except for the carbon atoms bonded to the cyclododecane and substituent(s) R₃, the other carbon atoms on the benzene ring are bonded to hydrogens, respectively; in the formula (1)b, except for the carbon atoms bonded to coronene and the substituent(s) R₃, the other carbon atoms on the benzene ring are bonded to hydrogens, respectively. Similarly, that i is an integer from 0 to 3 means that there may be 0 to 3 substituents R₃ on the benzene ring. In a case where there are a plurality of (more than one) substituents R₃, the plurality of substituents R₃ are present on different carbon atoms of the benzene ring. In a case where i is 0, the benzene ring is not substituted by the substituent R₃. In this case, in the formula (1)a, except for the carbon atoms bonded to the cyclododecane and the substituent(s) R₁, the other carbon atoms on the benzene ring are bonded to hydrogens, respectively; in the formula (1)b, except for the carbon atoms bonded to the coronene and the substituent(s) R₁, the other carbon atoms on the benzene ring are bonded to hydrogens, respectively.

In the formula (1)a, that n is an integer from 0 to 5 means that there may be 0 to 5 substituents R₂ on the cyclododecane. In a case where there are a plurality of (more than one) substituents R₂, the plurality of substituents R₂ are present on different carbon atoms of the cyclododecane. In a case where n is 0, the cyclododecane is not substituted by the substituent R₂. In this case, in the formula (1)a, except that m substituents R₁ are bonded to the corresponding number of carbon atoms and i substituents R₃ are bonded to the corresponding number of carbon atoms, the other carbon atoms are bonded to

hydrogens, respectively.

Z₁ to Z₁₁ are the same or different, and are each selected independently from any one of H and a substituent R. The substituent R, the substituent R₁ and the substituent R₂ are the same or different, and are each selected independently from any one of deuterium, halogen, cyano, nitryl, amino, C₁-C₄₀ alkyl, C₂-C₄₀ alkenyl, C₂-C₄₀ alkynyl, C₃-C₄₀ cycloalkyl, C₃-C₄₀ heterocycloalkyl, C₆-C₆₀ aryl, C₅-C₆₀ heteroaryl, C₁-C₄₀ alkoxy, C₆-C₆₀ aryloxy, C₃-C₄₀ alkylsilyl, C₆-C₆₀ arylsilyl, C₁-C₄₀ alkylboron, C₆-C₆₀ arylboron, C₆-C₆₀ arylphosphinylene, C₆-C₆₀ monoaryl or diaryl phosphino and C₆-C₆₀ arylamino; or, they may each be independently combined with an adjacent group to form a condensed ring.

The substituent R, the substituent R₁ and the substituent R₂ are combined with adjacent groups to form condensed rings, respectively, which means that the substituent

R, the substituent R₁ and the substituent R₂ can each be connected with an adjacent group to form a ring. Here, in an example where Z₉ in Z₁ to Z₁₁ is O, Z₉ is combined with an adjacent group to form a condensed ring, which may mean that 0 is combined with an adjacent carbon atom to form a condensed ring, so as to obtain the structure shown in the following formula (1)b-1.

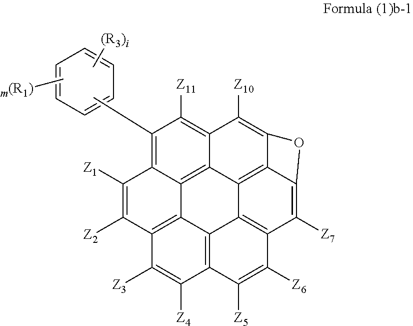

R₃ is selected from any one of substituted or unsubstituted heterocyclyl and fused heterocyclyl.

The heterocyclyl may be a five-membered heterocyclyl or a six-membered heterocyclyl. The five-membered heterocyclyl may be, for example, pyrrolyl, thiazolyl, imidazolyl, pyrazolyl, or furyl. The six-membered heterocyclyl may be, for example, pyridyl, pyrimidinyl, pyrazinyl, triazinyl, or pyranyl. The fused heterocyclyl may be indolyl, purinyl, quinolinyl, benzothiazolyl, carbazolyl, pteridyl, acridinyl, etc. These heterocyclic groups have good electron-withdrawing properties, and in a case where they are used as a hole blocking material, the light-emitting properties of the light-emitting devices can be improved.

In some embodiments, i is not 0, and R₃ is selected from any one of the following formulas (3)a, (3)b, (3)c, (3)d, and (3)e.

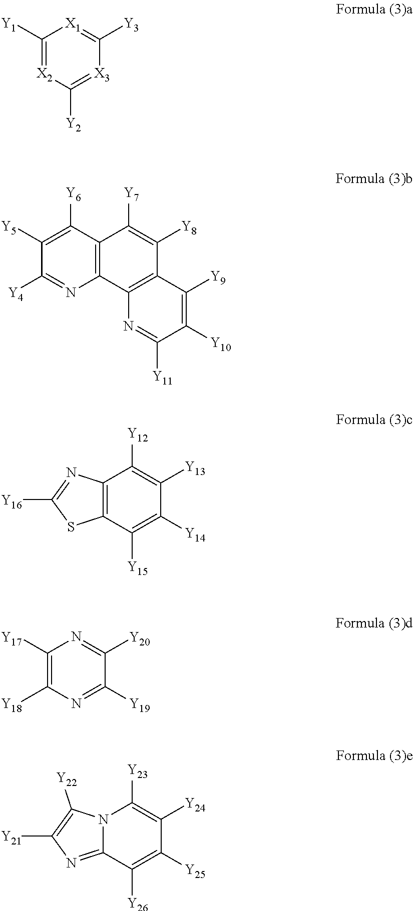

In the formula (3)a, X₁ to X₃ are each C(Y) or N, and at least two of them are N;

one of Y and Y₁ to Y₃ is combined with a dotted line in the formula (1)a or the formula (1)b, and the others are the same or different, and are each selected independently from any one of the hydrogen and the substituent R. In the formula (3)b, one of Y₄ to Y₁₁ is combined with the dotted line in the formula (1)a or the formula (1)b, and others are the same or different, and are each selected independently from any one of the hydrogen and the substituent R. In the formula (3)c, one of Y₁₂ to Y₁₆ is combined with the dotted line in the formula (1)a or the formula (1)b, and the others are the same or different, and are each selected independently from any one of the hydrogen and the substituent R. In the formula (3)d, one of Y₁₇ to Y₂₀ is combined with the dotted line in the formula (1)a or the formula (1)b, and the others are the same or different, and are each selected independently from any one of the hydrogen and the substituent R. In the formula (3)e, one of Y₂₁ to Y₂₆ is combined with the dotted line in the formula (1)a or the formula (1)b, and the others are the same or different, and are each selected independently from any one of the hydrogen and the substituent R.

According to a situation that X₁ to X₃ are each C(Y) or N and at least two of them are N in the formula (3)a, it can be known that the formula (3)a may be triazinyl (i.e., X₁ to X₃ are all N) or pyridazinyl (i.e., two of X₁ to X₃ are N). The formula (3)d is pyrazinyl. The formula (3)c and the formula (3)e are both fused heteroaryl. Each of the above formulas is a group with a strong electron-withdrawing capacity, so that the hole blocking layer 135 has good charge transport properties.

Considering the formula (3)c as an example, according to a situation that one of Y₁₂ to Y₁₆ is combined with the dotted line in the formula (1)a or the formula (1)b, and the others are the same or different, and are each selected independently from any one of the hydrogen and the substituent R, it can be seen that the formula (1)a may be shown as the following formula (1)a-1, and the formula (1)b may be shown as the following formula (1)b-2.

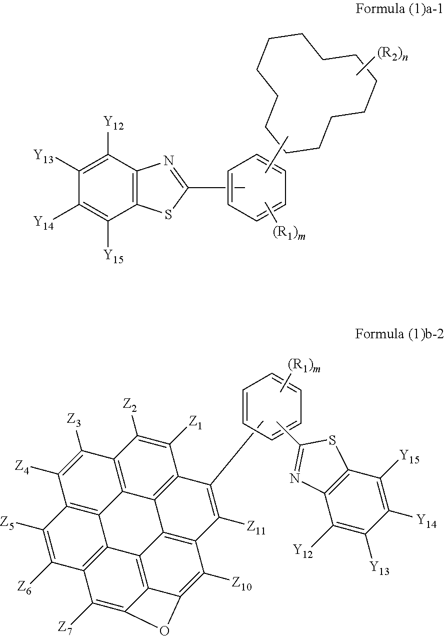

On a basis of the structures of the formula (1)a-1 and the formula (1)b-2, in a case where one of Y₁₂ and Y₁₃, e.g., Y₁₂, is selected from a substituent R that is combined with an adjacent group to form a condensed ring, the substituent R may be a substituent capable of condensing with an adjacent group to form a ring. For example, the substituent R may be valeric acid (CH₃CH₂CH₂CH₂COOH(C₅H₁₀O₂)), a group adjacent thereto may be C—Y₁₃, and Y₁₃ may be H. The substituent R and the C at a connection position of Y₁₃ are condensed into a ring, which may be expressed as the structure shown in the following formula (1)a-2 and formula (1)b-3.

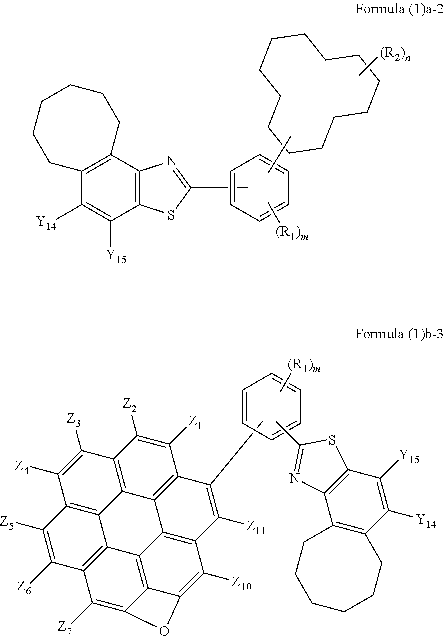

In the light-emitting device provided in the embodiments of the present disclosure, by introducing large groups such as the coronene and the cyclododecane into a bipolar compound with an electron-withdrawing group and an electron-donating group, the hole blocking material can have a high glass transition temperature. As a result, the hole blocking material has good film formation properties and excellent thermal stability. Meanwhile, by introducing the substituent R into a basic molecular skeleton, the highest occupied molecular orbital (HOMO) energy level and the lowest unoccupied molecular orbital (LUMO) energy level of the hole blocking material can be adjusted, so that the hole blocking material has good electron transport properties and hole blocking properties. Especially in a case where the substituent R is aryl or heteroaryl, a molecular weight of the molecule can be increased, thereby further increasing the glass transition temperature of the hole blocking material.

In this way, in a case where the hole blocking material provided in the embodiments of the present disclosure is used to form the hole blocking layer 135 in an organic light-emitting diode (OLED) device, a driving voltage of the OLED device can be reduced, and the light-emitting properties and the service life of the OLED device can be greatly improved.

In some embodiments, in the formula (1)a, at least m of m and n is not 0, and the substituent R₁ is selected from the structure shown in the following formula (2).

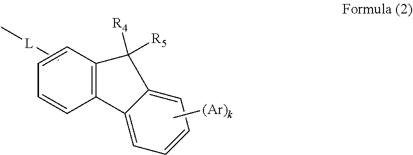

In the formula (2), R₄ and R₅ are the same or different, and are each selected independently from any one of deuterium, halogen, cyano, nitryl, C₁-C₄₀ alkyl, C₂-C₄₀ alkenyl, C₂-C₄₀ alkynyl, C₃-C₄₀ cycloalkyl, C₃-C₄₀ heterocycloalkyl, C₆-C₆₀ aryl, C₅-C₆₀ heteroaryl, C₁-C₄₀ alkoxy, C₆-C₆₀ aryloxy, C₃-C₄₀ alkylsilyl, C₆-C₆₀ arylsilyl, C₁-C₄₀ alkylboron, C₆-C₆₀ arylboron, C₆-C₆₀ arylphosphinylene, C₆-C₆₀ monoaryl or diaryl phosphino and C₆-C₆₀ arylamino; or, R₄ and R₅ are each independently combined with an adjacent group to form a condensed ring; k is an integer from 0 to 2, Ar is selected from any one of C₁-C₁₀ alkyl, C₃-C₁₀ cycloalkyl, and C₆-C₃₀ aryl, and L is selected from any one of a single bond, substituted or unsubstituted divalentaryl, and substituted or unsubstituted divalentheteroaryl.

At least m of m and n being not 0 has the following cases. In a first case, m is 1 or 2, and n is 0. In this case, the formula (1)a may be expressed as having the structure shown in the following formula (1)a-3. In a second case, both m and n are not 0. In this case, the substituent R₂ and the substituent R₁ may be the same or different. For example, in a case where the substituent R₁ is selected from the structure shown in the formula (2), the substituent R₂ is selected from the structure shown in the formula (2) or a structure different from the structure shown in the formula (2), such as benzene, or alkyl.

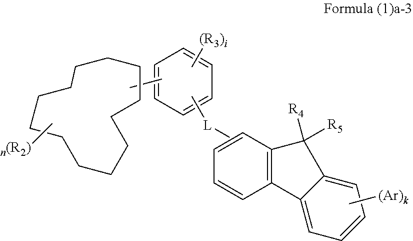

Here, it will be noted that, in a case where L is the single bond, the formula (1)a-3 may be expressed as follows.

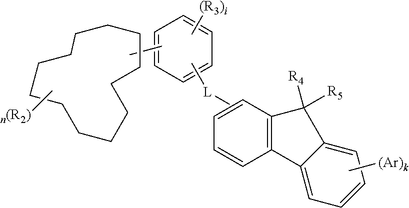

That k is an integer from 0 to 2 means that the number of substituents Ar on the benzene ring may be 0 to 2. In a case where k is 0, there is no substituent Ar on the benzene ring, and the carbon atoms on the benzene ring are bonded to hydrogens, respectively. In a case where k is 2, there are two substituents Ar on the benzene ring, and the two substituents Ar are present on different carbon atoms of the benzene ring.

In a case where m and n are both not 0, n may be greater than or equal to 1. It can be seen that, in a case where n is greater than 1, the substituents R₂ may be the same or different. In this case, there are many different cases depending on whether the substituents R₂ are the same as the substituent R₁. In a first case, the substituents R₂ are different from the substituent R₁. In this case, in the case where the substituent R₁ is selected from the structure shown in the formula (2), each substituent R₂ is different from the substituent R₁, and the substituents R₂ are the same or different, and are each selected independently from a structure different from the structure shown in the formula (2), such as benzene or alkyl. In a second case, a part of the plurality of substituents R₂ are the same as the substituent R₁, and the other part are different from the substituent R₁. In this case, in an example where n is 2 and the substituent R₁ is selected from the structure shown in the formula (2), in the two substituents R₂, one substituent R₂ is the same as the substituent R₁, and the other substituent R₂ is selected from a structure different from the structure shown in the formula (2), such as benzene or alkyl. In a third case, each substituent R₂ is the same as the substituent R₁. In the example where n is 2 and the substituent R₁ is selected from the structure shown in the formula (2), the two substituents R₂ are both selected from the structure shown in the formula (2).

In some embodiments, the material of the hole blocking layer 135 is selected from one or more of the structures shown in the following formula.

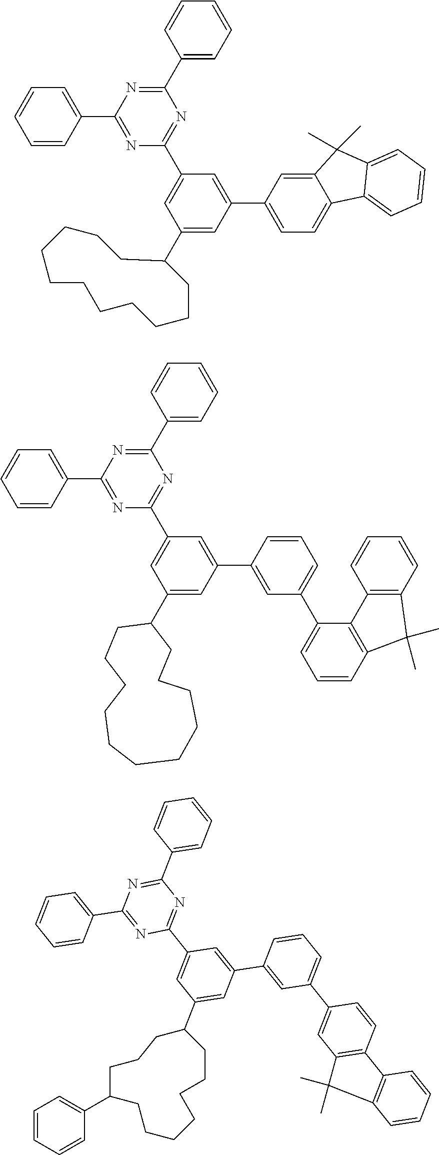

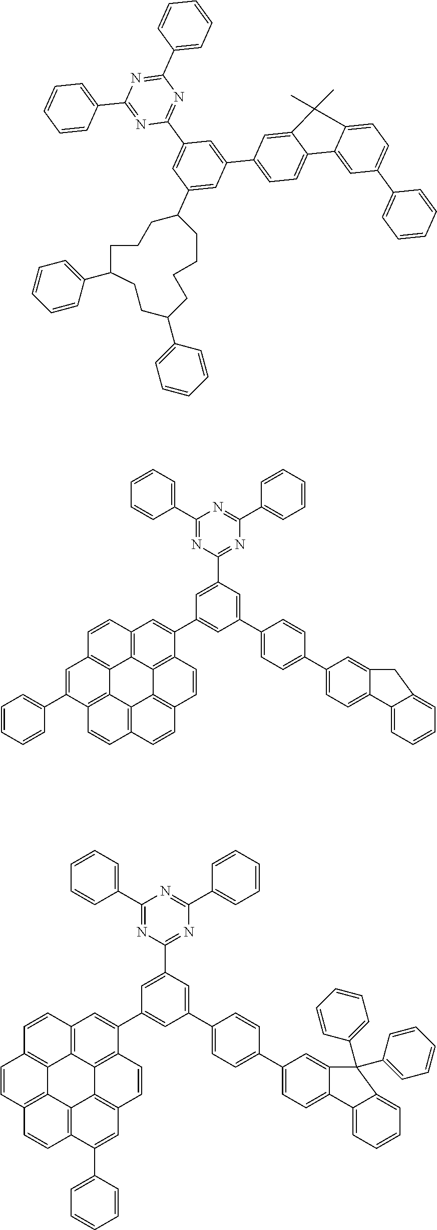 [SMILES: CC1(C)c2ccc(-c3ccccc3)cc2-c2ccc(-c3cc(-c4nc(-c5ccccc5)nc(-c5ccccc5)n4)cc(C4CCCCC(c5ccccc5)CCC(c5ccccc5)CCC4)c3)cc21 ; c1ccc(-c2nc(-c3ccccc3)nc(-c3cc(-c4ccc(-c5ccc6c(c5)Cc5ccccc5-6)cc4)cc(-c4cc5ccc6cc(-c7ccccc7)c7ccc8ccc9ccc4c4c9c8c7c6c54)c3)n2)cc1 ; c1ccc(-c2nc(-c3ccccc3)nc(-c3cc(-c4ccc(-c5ccc6c(c5)C(c5ccccc5)(c5ccccc5)c5ccccc5-6)cc4)cc(-c4cc5ccc6ccc7ccc8c(-c9ccccc9)cc9ccc4c4c5c6c7c8c94)c3)n2)cc1]

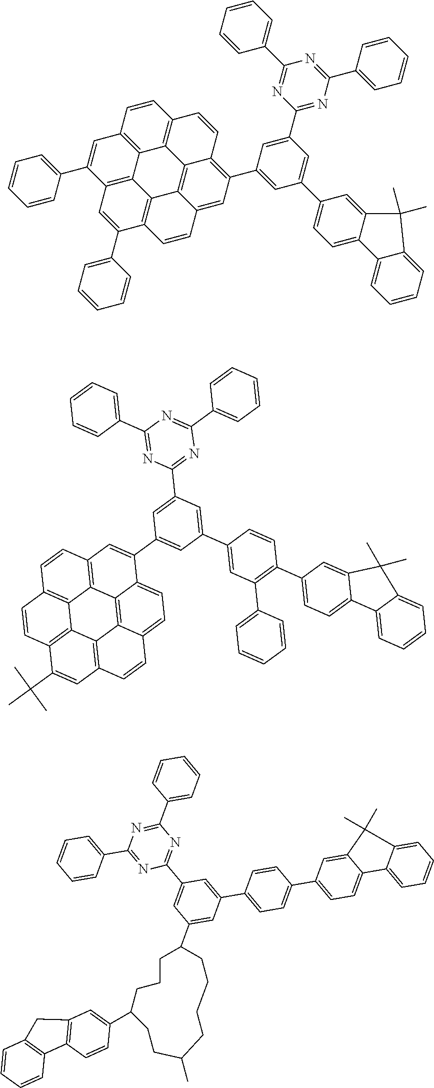

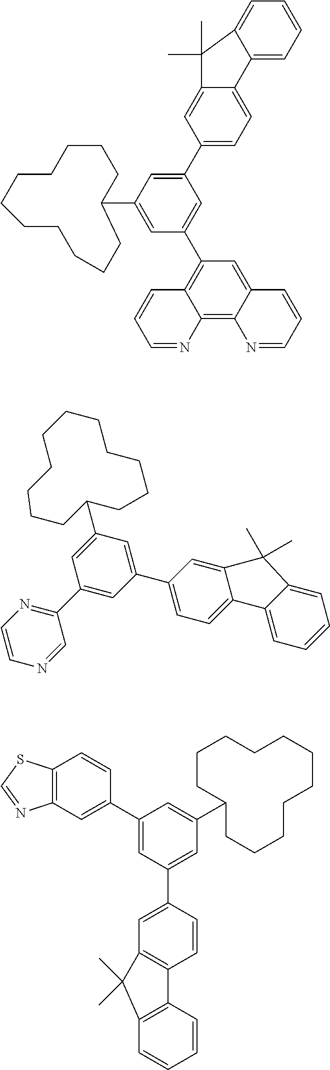 [SMILES: CC1(C)c2ccccc2-c2ccc(-c3cc(-c4cc5cccnc5c5ncccc45)cc(C4CCCCCCCCCCC4)c3)cc21 ; CC1(C)c2ccccc2-c2ccc(-c3cc(-c4cnccn4)cc(C4CCCCCCCCCCC4)c3)cc21 ; CC1(C)c2ccccc2-c2ccc(-c3cc(-c4ccc5scnc5c4)cc(C4CCCCCCCCCCC4)c3)cc21]

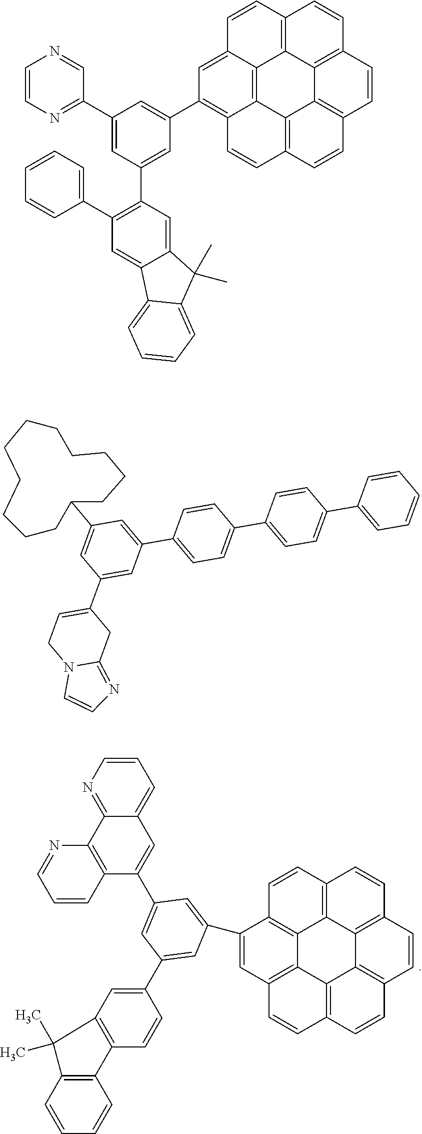 [SMILES: CC1(C)c2ccccc2-c2cc(-c3ccccc3)c(-c3cc(-c4cnccn4)cc(-c4cc5ccc6ccc7ccc8ccc9ccc4c4c9c8c7c6c54)c3)cc21 ; C1=C(c2cc(-c3ccc(-c4ccc(-c5ccccc5)cc4)cc3)cc(C3CCCCCCCCCCC3)c2)Cc2nccn2C1 ; CC1(C)c2ccccc2-c2ccc(-c3cc(-c4cc5cccnc5c5ncccc45)cc(-c4cc5ccc6ccc7ccc8ccc9ccc4c4c9c8c7c6c54)c3)cc21]

In some embodiments, the HOMO energy levels of the compounds containing the coronene or the cyclododecane are less than −5.6 eV. In a case where the compound is applied in the OLED device to be used to form the hole blocking layer 135, it has a good hole blocking capacity, which can solve a problem that the luminous efficiency is difficult to be improved due to an ineffective current flow (no light emission) caused by the imbalance transmission of the holes and the electrons.

In some embodiments, the HOMO energy level and the LUMO energy level of the compound containing the coronene or the cyclododecane meet the following formula:

|E_{HOMO}−E_{LUMO}|≥3.2 eV.

In the embodiments, the compound has a large band gap, so that the electrons and the holes can be combined in the light-emitting layer for recombining, thereby increasing a light-emitting region.

In some embodiments, the lowest singlet energy of the compounds containing the coronene or the cyclododecane are greater than 3.1 eV, and a difference between the lowest singlet energy and the lowest triplet energy of the compound containing the coronene or the cyclododecane is greater than 0.51 eV. The compound has a good exciton blocking capacity, which can confine singlet excitons and triplet excitons in the light-emitting layer, and improve the luminous efficiency.

In some embodiments, the glass transition temperature of the compound containing the coronene or the cyclododecane is within a range of 136° C. to 153° C., inclusive. The compound containing the coronene or the cyclododecane has a high glass transition temperature, which can improve film forming properties and thermal stability.

In some embodiments, an energy level difference between the HOMO energy level of the light-emitting layer 133 and the HOMO energy level of the hole blocking layer 135 is greater than 0.2 eV.

In the embodiments, a light-emitting material may be selected such that an energy level difference between the HOMO energy level of the light-emitting material and the HOMO energy level of the compound containing the coronene or the cyclododecane is greater than 0.2 eV. After the light-emitting device 13 is manufactured, the hole blocking layer 135 has a good hole blocking capacity, which can confine the holes in the light-emitting layer 133 for preventing the holes from combining with the electrons in the electron transport layer 134, thereby avoiding a problem that the luminous efficiency is difficult to be improved.

In some embodiments, an absolute value of an energy level difference between the LUMO energy level of the light-emitting layer 133 and the LUMO energy level of the hole blocking layer 135 is less than 0.3 eV.

In the embodiments, a light-emitting material may be selected such that an energy level difference between a LUMO energy level of the light-emitting material and the LUMO energy level of the compound containing the coronene or the cyclododecane is less than 0.3 eV. After the light-emitting device 13 is manufactured, the hole blocking layer 135 has good electron transport properties, which helps the holes and the electrons to be recombined in the light-emitting layer.

In some embodiments, an absolute value of an energy level difference between the LUMO energy level of the hole blocking layer 135 and the LUMO energy level of the electron transport layer 134 is less than 0.3 eV.

In the embodiments, an electron transport material may be selected such that an energy level difference between a LUMO energy level of the electron transport material and the LUMO energy level of the compound containing the coronene or the cyclododecane is less than 0.3 eV. After the light-emitting device 13 is manufactured, the hole blocking layer 135 has good electron transport properties, which is beneficial to increase a transmission rate of the electrons, and can further solve the problem of the imbalance transmission of the holes and the electrons.

In some embodiments, an absolute value of an energy level difference between the HOMO energy level of the hole blocking layer 135 and the HOMO energy level of the electron transport layer 134 is greater than 0.2 eV.

In the embodiments, an electron transport material may be selected such that an absolute value of an energy level difference between a HOMO energy level of the electron transport material and the HOMO energy level of the compound containing the coronene or the cyclododecane is greater than 0.2 eV. After the light-emitting device is manufactured, the electron transport layer 134 has good hole blocking properties, which can effectively confine the holes at a boundary of the hole blocking layer 135 and the light-emitting layer 133. As a result, the holes are prevented from combining with the electrons in the electron transport layer 134, and the problem that the luminous efficiency is difficult to be improved may be avoided.

In some other embodiments, a material of the electron transport layer 134 includes one or more of the compounds containing the coronene or the cyclododecane shown in the formula (1)a and the formula (1)b. In this case, the material of the electron transport layer 134 and the material of the hole blocking layer 135 may be the same or different.

In some embodiments, a difference between energy of the hole blocking layer 135 in the lowest singlet excited state and energy of the light-emitting layer 133 in the lowest singlet excited state is greater than 0.2 eV, and a difference between energy of the hole blocking layer 135 in the lowest triplet excited state and energy of the light- emitting layer 133 in the lowest triplet excited state is greater than 0.2 eV.

In the embodiments, the hole blocking layer 135 has a good exciton blocking capacity, and can confine the excitons in the light-emitting layer 133, thereby helping to improve the luminous efficiency.

In some embodiments, a material of the light-emitting layer 133 includes a host material and a guest material. The host material is selected from any one of anthracene, benzanthracene, benzophenanthrene and/or pyrene compounds and their derivatives, and atropisomers of these compounds and their derivatives. The guest material is selected from arylamino type compounds, and selected from aromatic anthracene amine, aromatic anthracene diamine, aromatic pyrene amine, aromatic pyrene diamine, aromatic cocory amine or aromatic cocory diamine. The material is not limited to the examples, and may include any well-known host and guest materials.

In some embodiments, the material of the electron transport layer 134 may be selected from any one of benzimidazole derivatives, triazine derivatives, pyrimidine derivatives, pyridine derivatives, pyrazine derivatives, quinoxaline derivatives, quinoline derivatives, diazole derivatives, aromatic ketones, lactams, borane, diazaphospholsecyclopentadiene derivatives and phosphine oxide derivatives.

In some embodiments, as shown in FIG. 2, the light-emitting device 13 further includes an electron injection layer (EIL) 137 disposed between the first electrode 131 and the electron transport layer 134, and a hole injection layer (HIL) 138 disposed between the second electrode 132 and the hole transport layer 136.

A material of the electron injection layer 137 may be selected from five-membered ring containing nitrogen derivatives and fluorenone, anthraquinone dimethane, diphenquinone, thiopyran dioxide, azole, diazole, triazole, imidazole, perylenetetracarboxylic acid, fluorenylenemethane, anthraquinodimethane, anthrone, etc. and their derivatives, but is not limited thereto. A material of the hole injection layer 138 may be selected from aromatic tertiary amine derivatives and phthalocyanine derivatives.

In some embodiments, as shown in FIG. 2, the light-emitting device 13 further includes an electron blocking layer (EBL) 139 disposed between the light-emitting layer 133 and the hole transport layer 136. A material of the electron blocking layer 139 may be selected from any one of aromatic amine derivatives, benzidine-type triphenylamine, styrylamine-type triphenylamine, and diamine-type triphenylamine.

In some embodiments of the present disclosure, a light-emitting substrate is provided. As shown in FIG. 3, the light-emitting substrate 1 may include a base 11, and a pixel define layer 12 and a plurality of light-emitting devices 13 that are disposed on the base 11. The pixel define layer 12 has a plurality of openings Q, and the plurality of light-emitting devices 13 may be arranged in one-to-one correspondence with the plurality of openings Q. The plurality of light-emitting devices 13 here may be all or some of the light-emitting devices 13 included in the light-emitting substrate 1. The plurality of openings Q may be all or some of the openings in the pixel define layer 12. At least one of the plurality of light-emitting devices 13 is a light-emitting device with the compound containing the coronene or the cyclododecane.

Depending on a situation that light-emitting colors of the plurality of light-emitting devices 13 in the light-emitting substrate 1 may be the same or different, the light-emitting substrate 1 may be an illumination substrate or a display substrate.

In a case where the light-emitting colors of the plurality of light-emitting devices 13 are the same, the hole blocking layer 139, the electron transport layer 134, the hole transport layer 136 and even the light-emitting layer 133 may all be arranged in a manner of a whole layer. In this case, each light-emitting device 13 is the light-emitting device with the compound containing the coronene or the cyclododecane.

In a case where the light-emitting colors of the plurality of light-emitting devices 13 are different, the electron transport layer 134 and the hole transport layer 136 are both arranged in a manner of a whole layer. The light-emitting layers 133 and the hole blocking layers 135 may be disposed in different openings Q according to different light-emitting colors of the light-emitting devices 13. In this case, the light-emitting layers 133 and the hole blocking layers 135 may each be formed through evaporation using a fine mask as a mask.

The light-emitting substrate 1 provided in the embodiments of the present disclosure has the same beneficial technical effects as the light-emitting device provided in the embodiments of the present disclosure, which will not be repeated here.

In some embodiments of the present disclosure, a light-emitting apparatus is provided, including the light-emitting substrate described above. Of course, the light-emitting apparatus may further include other components. For example, it may include a circuit for providing electrical signals to the light-emitting substrate to drive the light-emitting substrate to emit light. The circuit may be referred to as a control circuit, and may include a circuit board electrically connected to the light-emitting substrate and/or an integrated circuit (IC) electrically connected to the light-emitting substrate.

In some embodiments, the light-emitting apparatus may be an illumination apparatus. In this case, the light-emitting substrate may be the illumination substrate, for example, it may be served as a light source to achieve an illumination function. For example, the light-emitting substrate may be a backlight module in a liquid crystal display apparatus, a lamp for internal or external illumination, or a signal lamp.

In some other embodiments, the light-emitting apparatus may be a display apparatus. In this case, the light-emitting substrate is the display substrate, and is used to achieve a function of displaying images (i.e., pictures). The light-emitting apparatus may include a display or a product including the display. The display may be a flat panel display (FPD), or a micro display, etc. If classified according to whether users can see a scene behind the display, the display may be a transparent display or an opaque display. If classified according to whether the display can be bent or curled, the display may be a flexible display or a common display (which may be referred to as a rigid display). For example, the product including the display may be a computer display, a television, a billboard, a laser printer with a display function, a telephone, a mobile phone, a personal digital assistant (PDA), a laptop computer, a digital camera, a camcorder, a viewfinder, a vehicle, a large-area wall, a screen in a theater, or a sign in a stadium.

The light-emitting apparatus provided in the embodiments of the present disclosure has the same beneficial technical effects as the light-emitting device provided in the embodiments of the present disclosure, which will not be repeated here.

On this basis, in order to objectively evaluate the technical effects of the embodiments of the present disclosure, the technical solutions provided in the present disclosure will be exemplarily described below in detail through the following synthesis examples, experimental examples, application examples, and comparative examples.

### Synthesis Example 1

A synthesis of a compound 1.

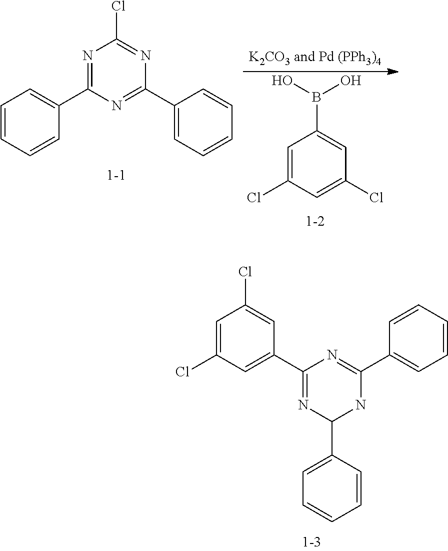 [SMILES: Clc1nc(-c2ccccc2)nc(-c2ccccc2)n1 ; OB(O)c1cc(Cl)cc(Cl)c1 ; Clc1cc(Cl)cc(C2=NC(c3ccccc3)NC(c3ccccc3)=N2)c1]

In step 1), 1-1, 1-2, K₂CO₃ and Pd(PPh₃)₄ are added in a mixed solution of dimethyl ether (DME) and water, and reflux is performed for about 12 hours under a protection of nitrogen. After being cooled to a room temperature (about 22° C.), the reaction mixture is filtered through a silica gel plug. An organic layer is separated, washed with water, and then dried over Na₂SO₄. After a solvent is evaporated, a crude product is purified through column chromatography on silica gel, and is eluted by using a mixed solvent of heptane and dichloromethane (a volume ratio of heptane to dichloromethane is within a range of 9/1 to 7/3) which is served as an eluent to obtain 1-3.

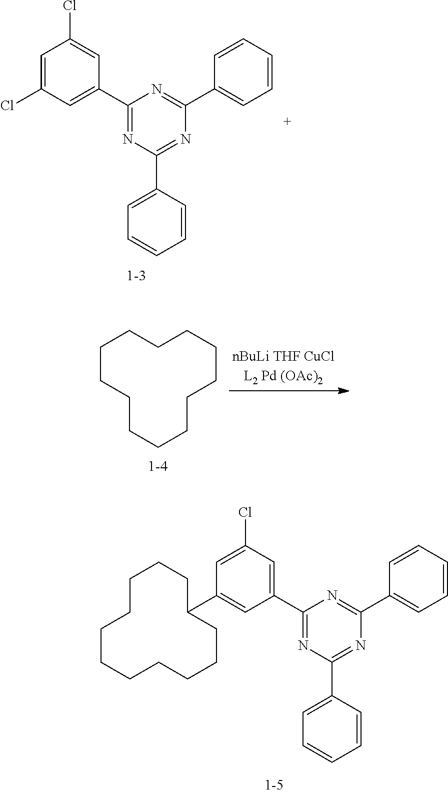 [SMILES: Clc1cc(Cl)cc(-c2nc(-c3ccccc3)nc(-c3ccccc3)n2)c1 ; C1CCCCCCCCCCC1 ; Clc1cc(-c2nc(-c3ccccc3)nc(-c3ccccc3)n2)cc(C2CCCCCCCCCCC2)c1]

In step 2), 1-4 is added to a three-necked flask, nitrogen is introduced, and then a certain amount of tetrahydrofuran is added; after the mixture is cooled to −80° C., an n-butyllithium ethane solution is slowly dripped and stirred. Then a cuprous chloride solution and a certain amount of palladium acetate, trimethoxy triphenylphosphine (L₂) and 1-3 that are soluble in the tetrahydrofuran are added and stirred at the room temperature, and then water and chloroform are added for extraction. The separated organic layer is dried, and column chromatography is performed on the separated organic layer, and the separated organic layer is recrystallized to obtain a product 1-5.

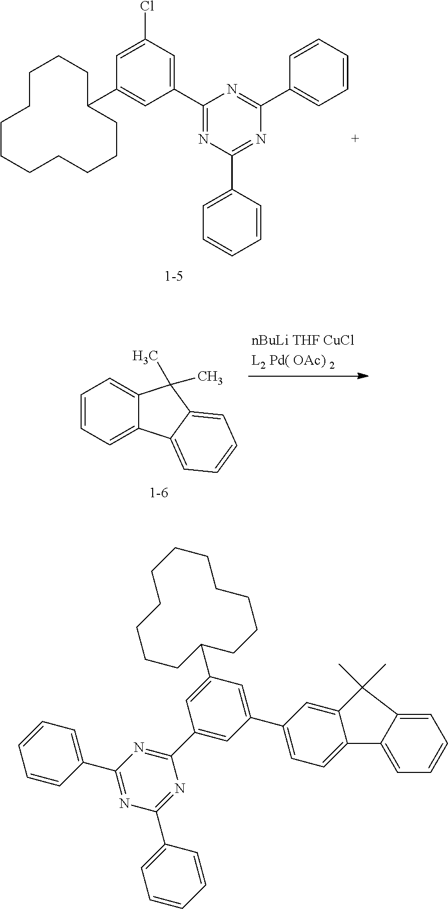

In step 3), 1-5 is added to a three-necked flask, nitrogen is introduced into the three-necked flask, and then a certain amount of tetrahydrofuran is added to the three-necked flask; after the mixture is cooled to −80° C., an n-butyllithium ethane solution is slowly dripped to the three-necked flask and stirred. Then the cuprous chloride solution and a certain amount of palladium acetate, trimethoxy triphenylphosphine (L₂) and 1-6 that are soluble in the tetrahydrofuran are added to the three-necked flask and stirred at the room temperature, and then water and chloroform are added to the three-necked flask for extraction. The separated organic layer is dried, and the column chromatography is performed on the separated organic layer, and the separated organic layer is recrystallized to obtain the compound 1.

The nuclear magnetic resonance (NMR) data of the compound 1 is: ¹³C-NMR: 174(s), 173.5(d), 152.3(d), 148.8(s), 144.6(s), 142.1(s), 139.1(d), 135.2(m), 134.5(s), 131.3(d), 129.2(m), 127.5(m), 125.9(m), 122.4(d), 119.8(d), 45.9(s), 41.7(s), 33.6(m), 30.8(m), 29.5 (d), 28.5(d), 26.3(d).

### Synthesis Example 2

A synthesis of a compound 2.

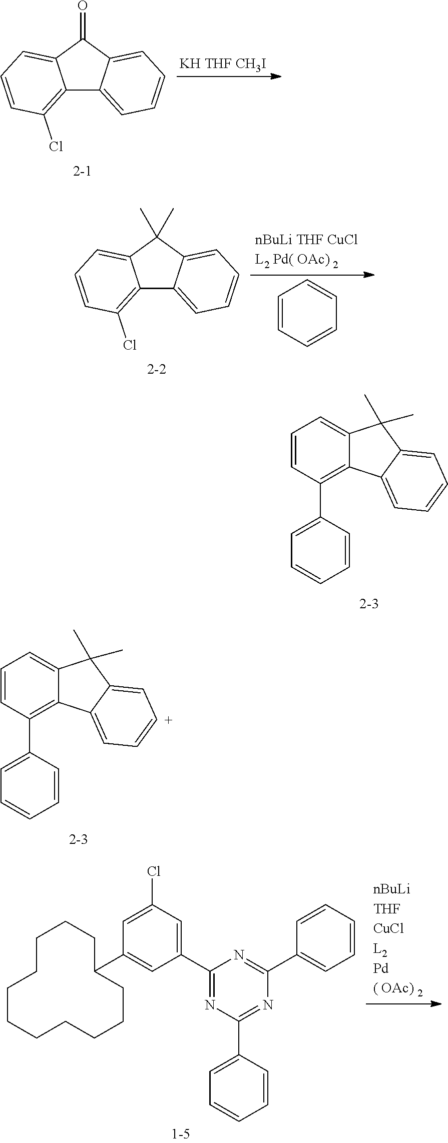

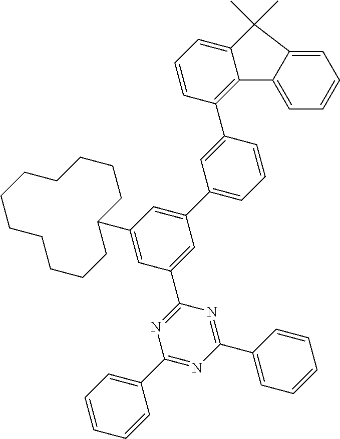 [SMILES: CC1(C)c2ccccc2-c2c(-c3cccc(-c4cc(-c5nc(-c6ccccc6)nc(-c6ccccc6)n5)cc(C5CCCCCCCCCCC5)c4)c3)cccc21]

Steps 1) and 2) are basically the same as the steps 1) and 2) for synthesizing the compound 1-5 in the synthesis example 1, and relevant chemical equations may be referred to that shown in step 1) and step 2) in the synthesis example 1. The difference is that, after synthesizing 1-5 and under an argon atmosphere, 35% potassiumhydride is added to anhydrous tetrahydrofuran (THF), and then fluorenone is added. After that, iodomethane is added, and a reaction occurs at a reflux temperature for 72 hours. Water is added to the obtained reaction mixture, and then dilute hydrochloric acid is added. The obtained mixture is extracted with chloroform, and the obtained extract is dried over anhydrous magnesium sulfate. A solvent is removed by reducing pressure, and the formed solid substance is separated through filtration, and is washed with methanol. The obtained substance is suspended in purified water, and ferric chloride monohydrate is added to the obtained suspension. After waiting for 1 hour at the room temperature, an aqueous solution obtained from chlorine and purified water (a volume ratio is 1 : 100) is dripped, and a reaction occurs at the room temperature for 12 hours. After separating the formed crystal by filtering, the crystal is washed with water and methanol and dissolved in chloroform; then the obtained solution is washed with a sodium bicarbonate aqueous solution and water and dried over anhydrous magnesium sulfate, and the solvent is removed through distillation. After hexane is added to the obtained mixture, 2-2 is formed through filtration and separation. Benzene is added to the three-necked flask, the nitrogen is introduced, and then a certain amount of tetrahydrofuran is added; after the mixture is cooled to −80° C., the n-butyllithium ethane solution is slowly dripped and stirred. Then a cuprous chloride solution and a certain amount of palladium acetate, trimethoxy triphenylphosphine (L₂) and 2-2 that are soluble in the tetrahydrofuran are added and stirred at the room temperature, and then water and chloroform are added for extraction. The separated organic layer is dried, and column chromatography is performed on the separated organic layer, and the separated organic layer is recrystallized to obtain a product 2-3.

The product 2-3 is added to another three-necked flask, nitrogen is introduced, and then a certain amount of tetrahydrofuran is added; after the mixture is cooled to −80° C., the n-butyllithium ethane solution is slowly dripped and stirred. Then a cuprous chloride solution and a certain amount of palladium acetate, trimethoxy triphenylphosphine (L₂) and 1-5 that are soluble in the tetrahydrofuran are added and stirred at the room temperature, and then water and chloroform are added for extraction. The separated organic layer is dried, and the column chromatography is performed on the separated organic layer, and the separated organic layer is recrystallized to obtain the compound 2.

The NMR data of the compound 2 is: ¹³C-NMR: 171.5(m), 154(s), 152.7(s), 150.9(s), 142.6(m), 137.4(m), 136.2(s), 134.9(d), 132.8(s), 131(d), 130.5(m), 129.9(m), 128.2(s), 127.6(m), 126.4(s), 125.5(d), 123.1(s), 121.1(d), 50.1(s), 44.5(s), 38.2(d), 28.8(m), 27.8(m), 27(d), 25.9(d).

### Synthesis Example 3

A synthesis of a compound 3.

Step 1) is basically the same as step 1) in the synthesis example 1, and will not be repeated here.

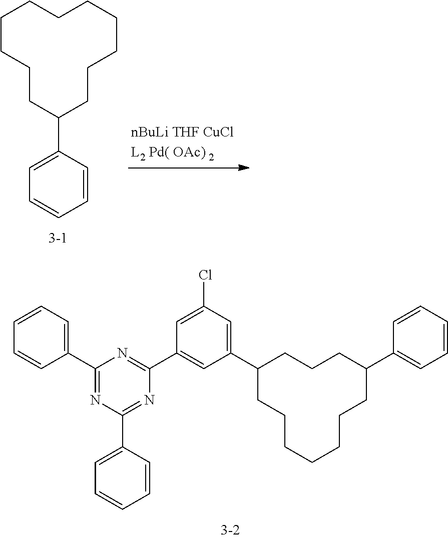

In step 2), 3-1 is added to a three-necked flask, nitrogen is introduced, and then a certain amount of tetrahydrofuran is added; after the mixture is cooled to −80° C., an n-butyllithium ethane solution is slowly dripped and stirred. Then the cuprous chloride solution and a certain amount of palladium acetate, trimethoxy triphenylphosphine (L₂) and 1-3 that is obtained in step 1) that are soluble in the tetrahydrofuran are added and stirred at the room temperature, and then water and chloroform are added for extraction. The separated organic layer is dried, and column chromatography is performed on the separated organic layer, and the separated organic layer is recrystallized to obtain a product 3-2.

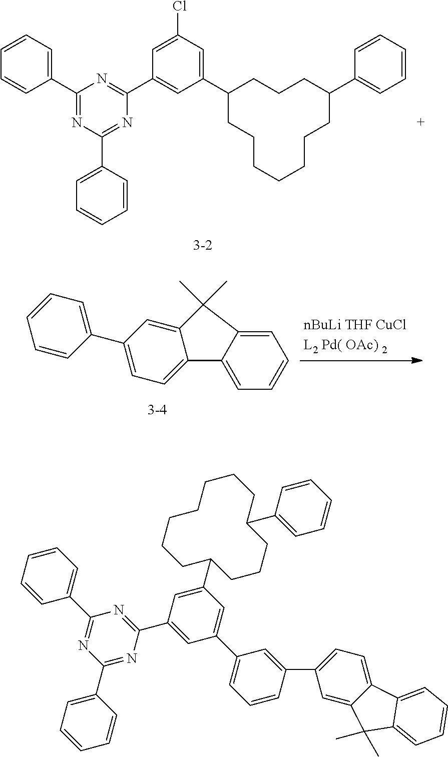

In step 3), 3-2 is added to a three-necked flask, nitrogen is introduced, and then a certain amount of tetrahydrofuran is added; after the mixture is cooled to −80° C., an n-butyllithium ethane solution is slowly dripped and stirred. Then the cuprous chloride solution and a certain amount of palladium acetate, trimethoxy triphenylphosphine (L₂) and 3-3 (CAS: 174753-91-4) that are soluble in the tetrahydrofuran are added and stirred at the room temperature, and then water and chloroform are added for extraction. The separated organic layer is dried, and the column chromatography is performed on the separated organic layer, and the separated organic layer is recrystallized to obtain the compound 3.

The NMR data of the compound 3 is: ¹³C-NMR: 172.2(d), 170.7(s), 147.8(d), 141(d), 139.9(d), 137.0(m), 134.7(d), 131.1(d), 130.5(m), 128.9(d), 127.5(m), 126.7(m), 123.2(d), 124.7(s), 121.6(s), 45.8(s), 39.3(s), 37.1(d), 30.9(d), 24.7(m), 21.8(d).

### Synthesis Example 4

A synthesis of a compound 4.

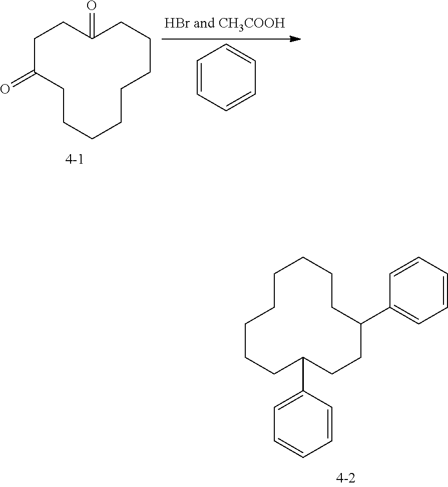

In step 1), 4-1, benzene, HBr and CH₃COOH are mixed in an aqueous solution, and reflux is performed for about 12 hours under the protection of nitrogen. After being cooled to a room temperature (about 22° C.), a reaction mixture is filtered through a silica gel plug. An organic layer is separated, washed with water, and then dried over Na₂SO₄. After a solvent is evaporated, a crude product is purified through the column chromatography on the silica gel, and is eluted by using the mixed solvent of heptane and dichloromethane (the volume ratio of heptane to dichloromethane is within the range of 9/1 to 7/3), which is served as the eluent, to obtain 4-2.

Step 2) for obtaining 1-3 is basically the same as step 1) in the synthesis example 1, and will not be repeated here.

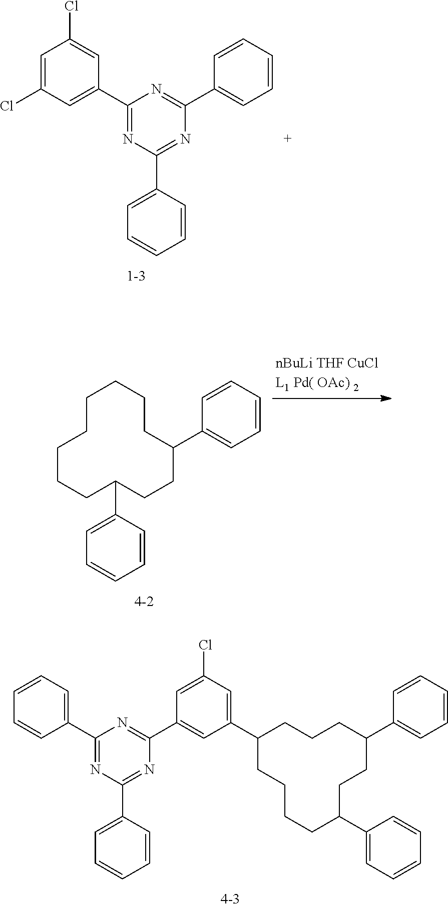

In step 3), 4-2 is added to a three-necked flask, nitrogen is introduced, and then a certain amount of tetrahydrofuran is added; after the mixture is cooled to −80° C., an n-butyllithium ethane solution is slowly dripped and stirred. Then a cuprous chloride solution and a certain amount of palladium acetate, trimethoxy triphenylphosphine (L₂) and 1-3 that are soluble in the tetrahydrofuran are added and stirred at the room temperature, and then water and chloroform are added for extraction. The separated organic layer is dried, and column chromatography is performed on the separated organic layer, and the separated organic layer is recrystallized to obtain a product 4-3.

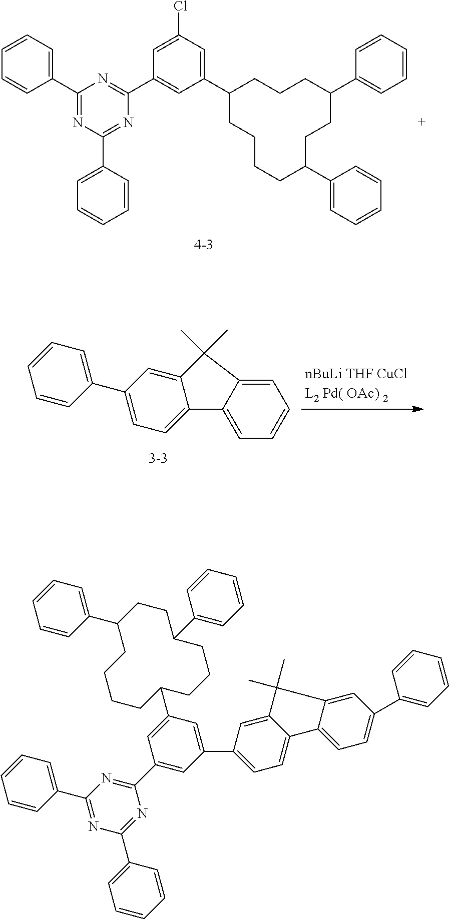

In step 4), 4-3 is added to a three-necked flask, nitrogen is introduced, and then a certain amount of tetrahydrofuran is added; after the mixture is cooled to −80° C., an n-butyllithium ethane solution is slowly dripped and stirred. Then a cuprous chloride solution and a certain amount of palladium acetate, trimethoxy triphenylphosphine (L₂) and 3-3 that are soluble in the tetrahydrofuran are added and stirred at the room temperature, and then water and chloroform are added for extraction. The separated organic layer is dried, and the column chromatography is performed on the separated organic layer, and the separated organic layer is recrystallized to obtain the compound 4.

The NMR data of the compound 4 is: ¹³C-NMR: 172.2(d), 170.7(s), 147.8(d), 141.0(d), 139.9(d), 137.0(m), 134.7(d), 131.1(d), 130.5(m), 129.2 (m), 128.9(d), 127.5(m), 126.7(m), 124.7(s), 123.2(d), 121.6(s), 45.8(s), 39.3(s), 37.1(d), 30.9 (d), 24.4(m), 21.8(d).

### Synthesis Example 5

A synthesis of a compound 5.

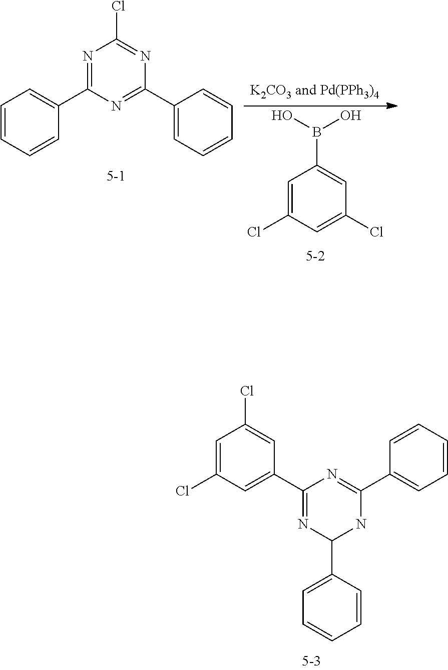 [SMILES: Clc1nc(-c2ccccc2)nc(-c2ccccc2)n1 ; OB(O)c1cc(Cl)cc(Cl)c1 ; Clc1cc(Cl)cc(C2=NC(c3ccccc3)NC(c3ccccc3)=N2)c1]

In step 1), 5-1, 5-2, K₂CO₃ and Pd(PPh₃)₄ are mixed in a solution (water bath) of DME and water, and reflux is performed for about 12 hours under the protection of nitrogen. After being cooled to a room temperature (about 22° C.), a reaction mixture is filtered through a silica gel plug. An organic layer is separated, washed with water, and then dried over Na₂SO₄. After a solvent is evaporated, a crude product is purified through the column chromatography on the silica gel, and is eluted by using the mixed solvent of heptane and dichloromethane (the volume ratio of heptane to dichloromethane is within the range of 9/1 to 7/3), which is served as the eluent, to obtain 5-3.

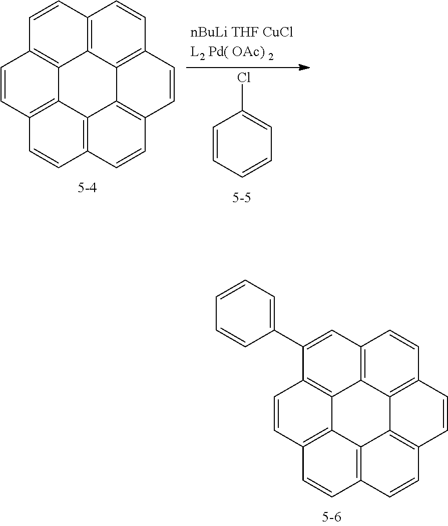

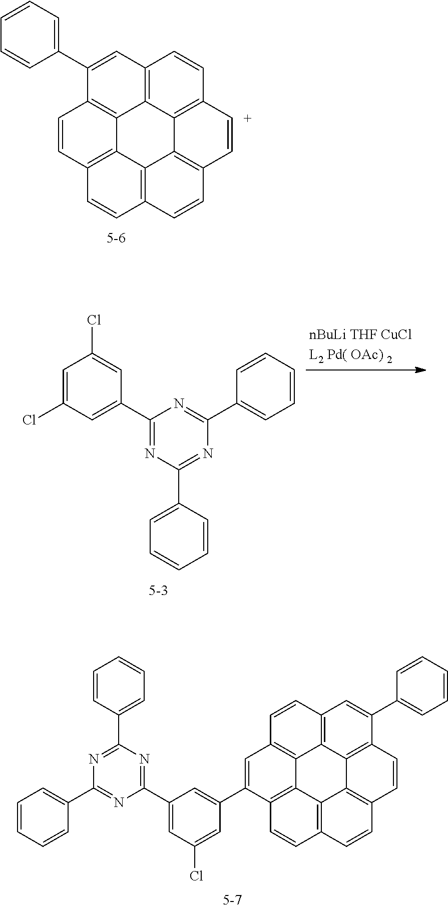

In step 2), 5-4 is added to a three-necked flask, nitrogen is introduced, and then a certain amount of tetrahydrofuran is added; after the mixture is cooled to −80° C., an n-butyllithium ethane solution is slowly dripped and stirred. Then the cuprous chloride solution and a certain amount of palladium acetate, trimethoxy triphenylphosphine (L₂) and 5-5 that are soluble in the tetrahydrofuran are added and stirred at the room temperature, and then water and chloroform are added for extraction. The separated organic layer is dried, and column chromatography is performed on the separated organic layer, and the separated organic layer is recrystallized to obtain a product 5-6. The product 5-6 is added to a three-necked flask, the nitrogen is introduced, and then a certain amount of tetrahydrofuran is added; after the mixture is cooled to −80° C., the n-butyllithium ethane solution is slowly dripped and stirred. Then a cuprous chloride solution and a certain amount of palladium acetate, trimethoxy triphenylphosphine (L₂) and 5-3 that are soluble in the tetrahydrofuran are added and stirred at the room temperature, and then water and chloroform are added for extraction. The separated organic layer is dried, and column chromatography is performed on the separated organic layer, and the separated organic layer is recrystallized to obtain a product 5-7.

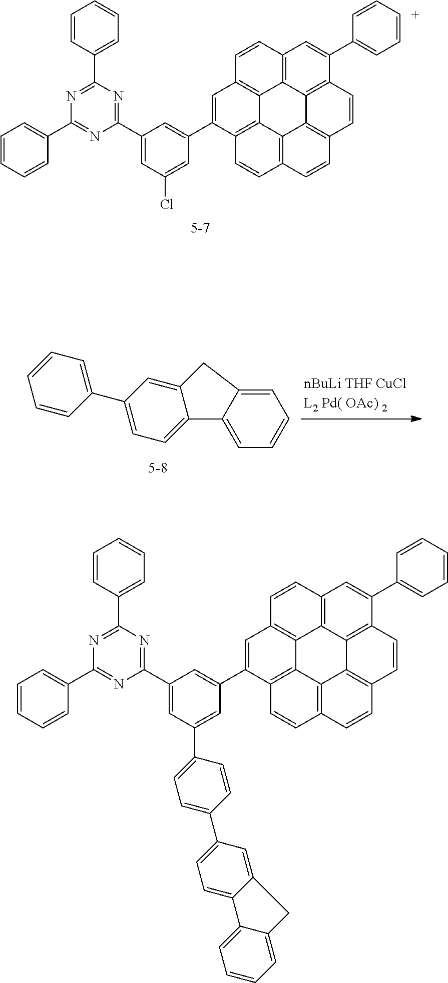

In step 3), 5-8 is added to a three-necked flask, nitrogen is introduced, and then a certain amount of tetrahydrofuran is added; after the mixture is cooled to −80° C., an n-butyllithium ethane solution is slowly dripped and stirred. Then a cuprous chloride solution and a certain amount of palladium acetate, trimethoxy triphenylphosphine (L₂) and 5-7 that are soluble in the tetrahydrofuran are added and stirred at the room temperature, and then water and chloroform are added for extraction. The separated organic layer is dried, and the column chromatography is performed on the separated organic layer, and the separated organic layer is recrystallized to obtain the compound 5.

The NMR data of the compound 5 is: ¹³C-NMR: 171.7(m), 145(s), 144.2(s), 143.6(s), 142.2(m), 141.3(d), 140.5(d), 139.4(d), 135.4(d), 134.7(d), 132.5(s), 131.1(m), 130.5(m), 129(m), 128.4(d), 127.6(m), 126.7(m), 126(m), 125(m), 124.6(m), 119.6(m), 118.8(m), 34.7(s).

### Synthesis Example 6

A synthesis of a compound 6.

Step 1) for obtaining 5-3 is basically the same as step 1) in the synthesis example 5, which will not be repeated here.

Step 2) for obtaining 5-7 is basically the same as step 2) in the synthesis example 5, which will not be repeated here.

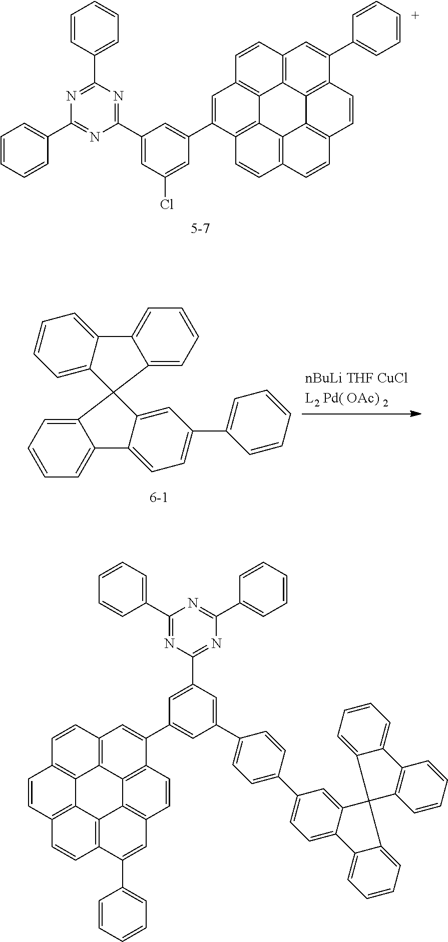

In step 3), 6-1 is added to a three-necked flask, nitrogen is introduced, and then a certain amount of tetrahydrofuran is added; after the mixture is cooled to −80° C., an n-butyllithium ethane solution is slowly dripped and stirred. Then a cuprous chloride solution and a certain amount of palladium acetate, trimethoxy triphenylphosphine (L₂) and 5-7 that are soluble in the tetrahydrofuran are added and stirred at the room temperature, and then water and chloroform are added for extraction. The separated organic layer is dried, and the column chromatography is performed on the separated organic layer, and the separated organic layer is recrystallized to obtain the compound 6.

The NMR data of the compound 6 is: ¹³C-NMR: 172.2(d), 170.7(s), 147.7(d), 142.4(d), 141.1(s), 193.9(s), 137.5(d), 137(d), 134.7(d), 134.1(m), 33.5(s), 131.9(m), 131.1(d), 130.5(s), 129.2(m), 128.1(s), 127.5(m), 126.9(m), 128.9(d), 127.6(d), 126.5(d), 124(s), 121.7(m), 120.0(s), 63.2(s).

### Synthesis Example 7

A synthesis of a compound 7.

Step 1) for obtaining 5-3 is basically the same as step 1) in the synthesis example 5, which will not be repeated here.

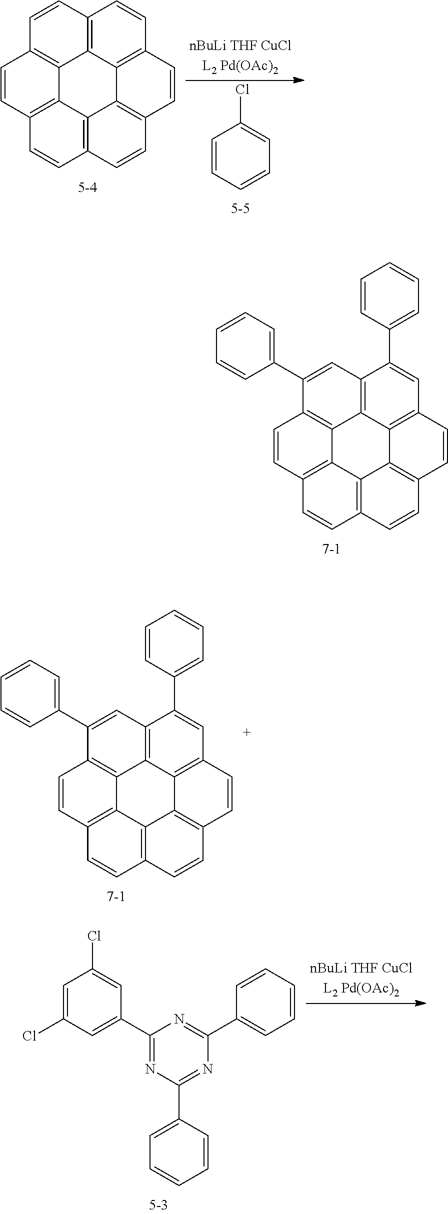

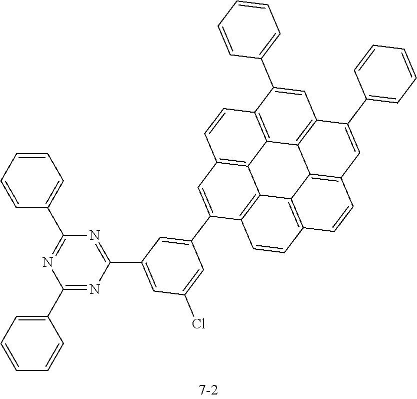 [SMILES: Clc1cc(-c2nc(-c3ccccc3)nc(-c3ccccc3)n2)cc(-c2cc3ccc4c(-c5ccccc5)cc5c(-c6ccccc6)cc6ccc7ccc2c2c7c6c5c4c32)c1]

In step 2), 5-4 is added to a three-necked flask, nitrogen is introduced, and then a certain amount of tetrahydrofuran is added; after the mixture is cooled to −80° C., an n-butyllithium ethane solution is slowly dripped and stirred. Then the cuprous chloride solution and a certain amount of palladium acetate, trimethoxy triphenylphosphine (L₂) and 5-5 that are soluble in the tetrahydrofuran are added and stirred at the room temperature, and then water and chloroform are added for extraction. The separated organic layer is dried, and column chromatography is performed on the separated organic layer, and the separated organic layer is recrystallized to obtain a product 7-1. The product 7-1 is added to a three-necked flask, nitrogen is introduced, and then a certain amount of tetrahydrofuran is added; after the mixture is cooled to −80° C., an n-butyllithium ethane solution is slowly dripped and stirred. Then a cuprous chloride solution and a certain amount of palladium acetate, trimethoxy triphenylphosphine (L₂) and 5-3 that are soluble in the tetrahydrofuran are added and stirred at the room temperature, and then water and chloroform are added for extraction. The separated organic layer is dried, and the column chromatography is performed on the separated organic layer, and the separated organic layer is recrystallized to obtain a product 7-2.

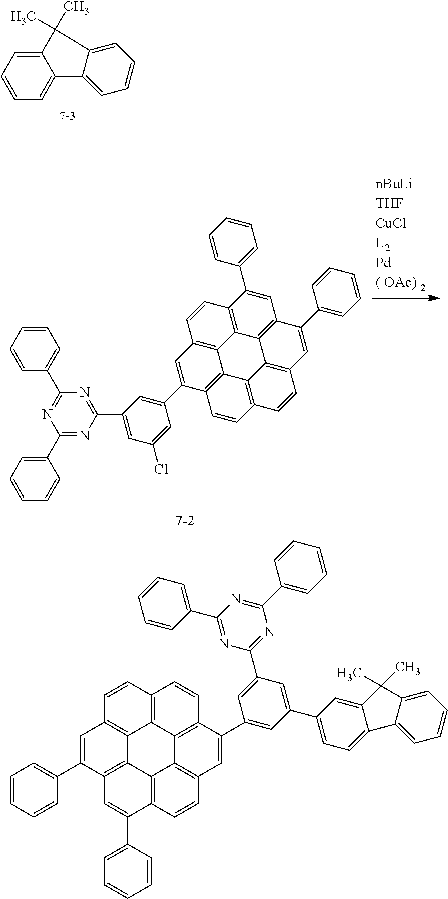

In step 3), 7-3 is added to a three-necked flask, nitrogen is introduced, and then a certain amount of tetrahydrofuran is added; after the mixture is cooled to −80° C., an n-butyllithium ethane solution is slowly dripped and stirred. Then a cuprous chloride solution and a certain amount of palladium acetate, trimethoxy triphenylphosphine (L₂) and 7-2 that are soluble in the tetrahydrofuran are added and stirred at the room temperature, and then water and chloroform are added for extraction. The separated organic layer is dried, and the column chromatography is performed on the separated organic layer, and the separated organic layer is recrystallized to obtain the compound 7.

The NMR data of the compound 7 is: ¹³C-NMR: 172.2(d), 170.7(s), 147.8(s), 141.0(m), 142(d), 137.5(m), 134.7(m), 133.5(m), 131.6(s), 131.1(d), 129.2(m), 127.6(m), 126.9(m), 126.2(s), 125.1(d), 124.7(m), 124(s), 123.2(d), 121.7(m), 120.0(d), 118.4(s), 42.9(s), 31.2(d).

### Synthesis Example 8

A synthesis of a compound 8.

Step 1) for obtaining 5-3 is basically the same as step 1) in the synthesis example 5, which will not be repeated here.

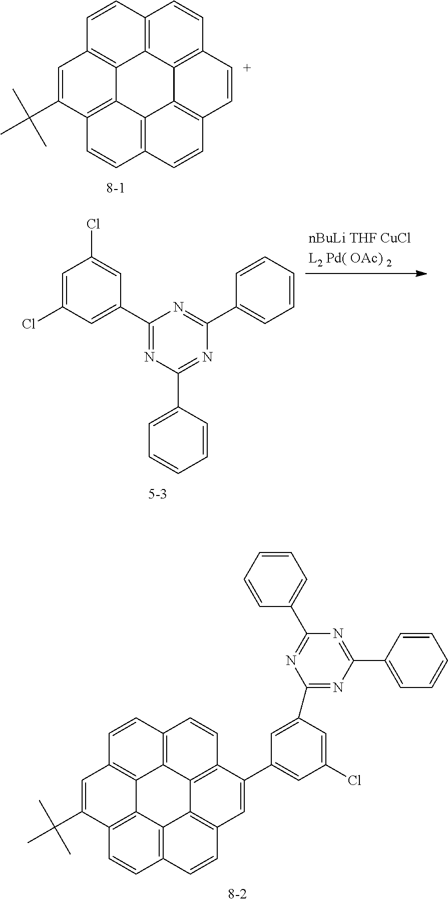

In step 2), 8-1 is added to a three-necked flask, nitrogen is introduced, and then a certain amount of tetrahydrofuran is added; after the mixture is cooled to −80° C., an n-butyllithium ethane solution is slowly dripped and stirred. Then a cuprous chloride solution and a certain amount of palladium acetate, trimethoxy triphenylphosphine (L₂) and 5-3 that are soluble in the tetrahydrofuran are added and stirred at the room temperature, and then water and chloroform are added for extraction. The separated organic layer is dried, and column chromatography is performed on the separated organic layer, and the separated organic layer is recrystallized to obtain a product 8-2.

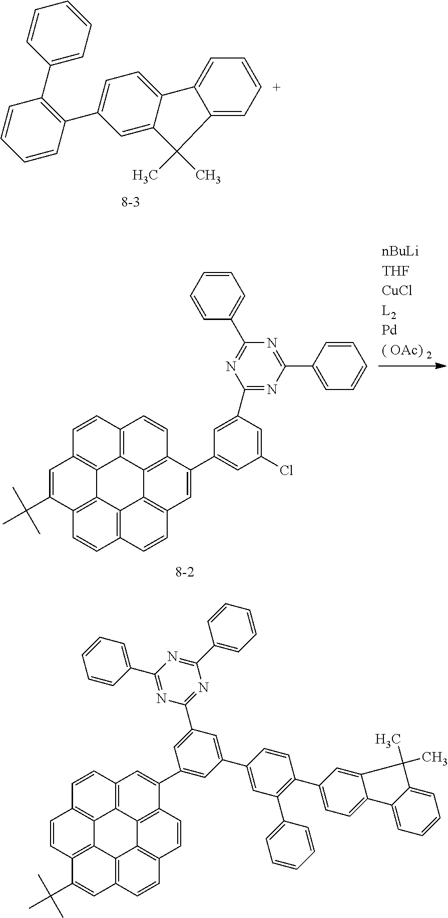

In step 3), 8-3 is added to a three-necked flask, nitrogen is introduced, and then a certain amount of tetrahydrofuran is added; after the mixture is cooled to −80° C., an n-butyllithium ethane solution is slowly dripped and stirred. Then a cuprous chloride solution and a certain amount of palladium acetate, trimethoxy triphenylphosphine (L₂) and 8-2 that are soluble in the tetrahydrofuran are added and stirred at the room temperature, and then water and chloroform are added for extraction. The separated organic layer is dried, and the column chromatography is performed on the separated organic layer, and the separated organic layer is recrystallized to obtain the compound 8.

The NMR data of the compound 8 is: ¹³C-NMR: 172.2(d), 170.7(s), 148.3(s), 147.8(s), 143.2(s), 142.0(s), 141.0(d), 139.9(s), 137.5(d), 136.0(d), 134.1(m), 133.5(s), 131.6(s), 131.1(d), 130.5(m), 129.2(m), 128.9(m), 127.5(m), 126.5(m), 125.5(d), 124.7(m), 123.2(s), 122.0(s), 121.6(d), 120(d), 45.8(s), 35.1(s), 31.7(m), 30.9(d).

### Experimental Example 1

HOMO energy levels, LUMO energy levels, singlet exciton energy, triplet exciton energy and glass transition temperatures of the compound 1 to the compound 8 obtained through the syntheses are tested, and data shown in Table 1 below are obtained through calculations.

TABLE 1

| | | \|E_{HOMO} − | | | |
|---|---|---|---|---|---|
| Compound | HOMO | E_{LUMO}\| | S1 | ΔEst | Tg/° C. |
| 1 | −5.8 | 3.6 | 3.42 | 0.51 | 136 |
| 2 | −5.9 | 3.7 | 3.59 | 0.60 | 142 |
| 3 | −6.0 | 3.5 | 3.52 | 0.68 | 140 |
| 4 | −5.9 | 3.5 | 3.43 | 0.65 | 143 |
| 5 | −5.7 | 3.5 | 3.13 | 0.52 | 146 |
| 6 | −5.8 | 3.4 | 3.11 | 0.55 | 144 |
| 7 | −5.6 | 3.2 | 3.11 | 0.56 | 151 |
| 8 | −5.6 | 3.2 | 3.14 | 0.53 | 153 |

It can be seen from Table 1 that, by introducing the coronene or the cyclododecane into a hole blocking material with endothelial differentiation genes (EDG) and electron-withdrawing groups (EWG), the hole blocking material can have a high glass transition temperature, so that the hole blocking material can have the good film formation properties and excellent thermal stability. In addition, by introducing the substituent R into the basic molecular skeleton and adjusting the substituent R, the HOMO energy level and the LUMO energy level of the hole blocking material can be adjusted to match the HOMO energy levels and LUMO energy levels of the adjacent layers, so as to improve the hole blocking effect, and enable the hole blocking material to have a large band gap. As a result, the electrons and the holes can be confined in the light-emitting layer for recombining, which increases the light-emitting region. In addition, through testing, it is found that the hole blocking material has high lowest singlet energy and lowest triplet energy, so that the hole blocking material has a good exciton blocking capacity. In a case where the hole blocking material is used in the hole blocking layer, the singlet excitons and the triplet excitons can be confined in the light-emitting layer, thereby improving the light-emitting efficiency of the device.

### Application Example

In the application example, an OLED device is provided. A structure of the OLED device is an indium tin oxide (ITO), HIL (which is made of HIA, and a thickness thereof is 20 nm), an HTL (which is made of HAT, and a thickness thereof is 20 nm), an auxiliary light-emitting layer (which is made of HTA, and a thickness thereof is 6 nm), a light-emitting layer (which is made of host material Host and 5% guest material Dopant, and a thickness thereof is 20 nm), an HBL (a thickness thereof is 50 nm), an ETL with 50 aluminum tris-(8-hydroxyquinoline) (AlQ₃) (a thickness thereof is 30 nm), an EIL (which is made of LiF, and a thickness thereof is 1 nm) and an Al cathode (a thickness thereof is 100 nm).

Molecular structures of H IA (i.e., N₂′,N_{7′,10}-triphenyl-N₂′,N₇′-bis(9-phenyl-9H-carbazol-3-yl) -10H-spiro[acridine-9,9′-fluorene]-2′,7′-diamine), HAT (i.e., (3,6,7,10,11-pentakis(aminomethyl) -4b,8a,8b,12a-tetrahydrodipyrazino[2,3-f:2′,3′-h]quinoxaline-2-carbonitrile), HTA (i.e., N([1,1′-biphenyl]-4-yl)-9,9-dimethyl-N-(4′-(7-phenyl-7H-benzo[c]carbazol-10-yl)-[1,1′-biphenyl]-4-yl)-9H-fluoren-2-amine), Host, Dopant and ETL are shown below. The HBL is selected from one of the compound 1 to the compound 8 provided in the synthesis examples, and the obtained devices are denoted as a device 1 to a device 8 in one-to-one correspondence.

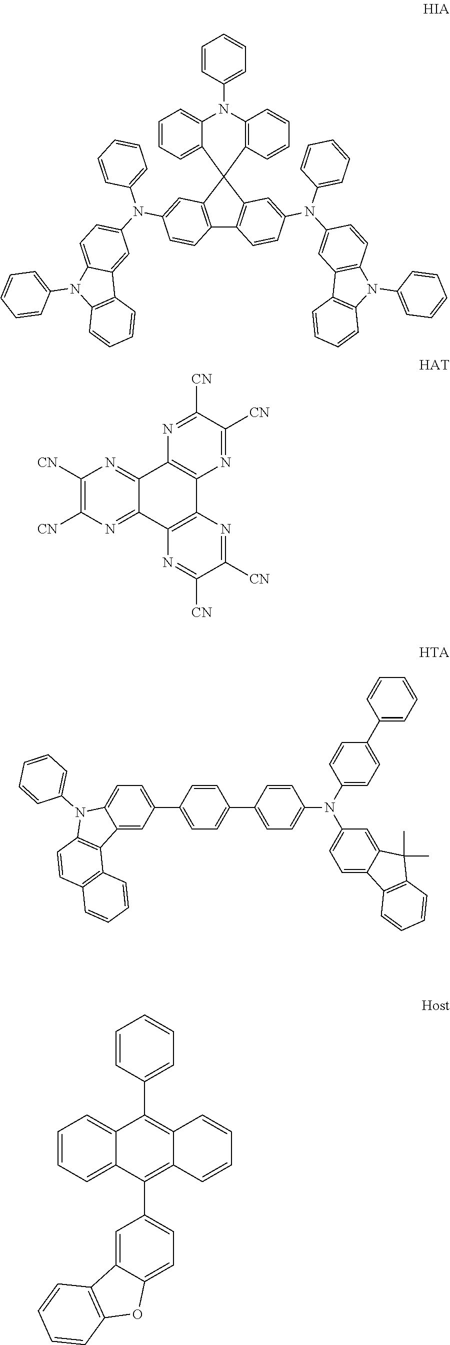

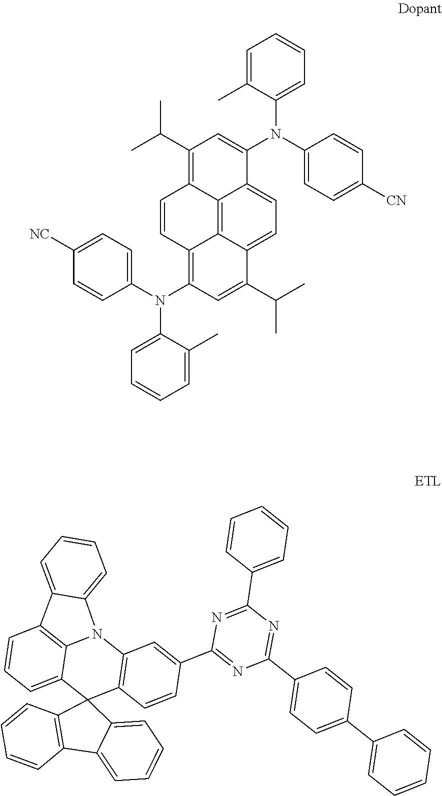 [SMILES: Cc1ccccc1N(c1ccc(C#N)cc1)c1cc(C(C)C)c2ccc3c(N(c4ccc(C#N)cc4)c4ccccc4C)cc(C(C)C)c4ccc1c2c43 ; c1ccc(-c2ccc(-c3nc(-c4ccccc4)nc(-c4ccc5c(c4)-n4c6ccccc6c6cccc(c64)C54c5ccccc5-c5ccccc54)n3)cc2)cc1]

### Comparative Example

A device provided in the comparative example has a structure the same as the structure of the device of the application example, and the difference is that the HBL adopts the structure shown in the following formula (HBL1). The device provided in the comparative example is denoted as a device 9.

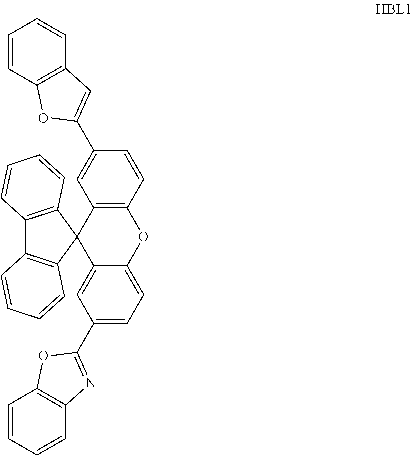 [SMILES: c1ccc2c(c1)-c1ccccc1C21c2cc(-c3cc4ccccc4o3)ccc2Oc2ccc(-c3nc4ccccc4o3)cc21]

### Experimental Example 2

Currents with a same density are supplied to the device 1 to the device 9, respectively, and driving voltages, service lives and current efficiencies of the device 1 to the device 9 are tested, so that data shown in Table 2 below are obtained.

TABLE 2

| | | Driving | Service | Current |
|---|---|---|---|---|
| | | voltage | life | efficiency |
| Sample | HBL | (V) | (T90/h) | (cd/A) |
| Device 1 | Compound 1 | 4.88 | 235 | 4.02 |
| Device 2 | Compound 2 | 4.61 | 210 | 4.11 |
| Device 3 | Compound 3 | 4.32 | 203 | 3.98 |
| Device 4 | Compound 4 | 4.50 | 190 | 4.09 |
| Device 5 | Compound 5 | 4.95 | 126 | 3.97 |
| Device 6 | Compound 6 | 4.88 | 156 | 3.88 |
| Device 7 | Compound 7 | 4.67 | 179 | 4.01 |
| Device 8 | Compound 8 | 4.49 | 115 | 3.80 |
| Device 9 | HBL1 | 4.81 | 70 | 3.78 |

It can be seen from Table 2 that, compared with the hole blocking material that only contains EDG and EWG, by introducing the coronene or the cyclododecane into the hole blocking material, the driving voltage can be reduced, the current efficiency can be increased, and the service life of the device can be prolonged.

In summary, by introducing larger groups such as the coronene or the cyclododecane into the hole blocking material with EDG and EWG, the hole blocking material can have a higher glass transition temperature, so that the hole blocking material can have the good film formation property and excellent thermal stability. Meanwhile, by introducing the substituent R into the basic molecular skeleton, the HOMO energy level and LUMO energy level of the hole blocking material can be adjusted, so that the hole blocking material can have good electron transporting properties and hole blocking properties. Meanwhile, in a case of applying the hole blocking material to the light-emitting device as a hole blocking material and/or an electron transport material, the service life of the light-emitting device can be prolonged in a case of maintaining a low driving voltage and a high current efficiency.

The foregoing descriptions are merely specific implementation manners of the present disclosure, but the protection scope of the present disclosure is not limited thereto. Any person skilled in the art could conceive of changes or replacements within the technical scope of the present disclosure, which shall be included in the protection scope of the present disclosure. Therefore, the protection scope of the present disclosure shall be subject to the protection scope of the claims.

1. A light-emitting device, comprising:
a first electrode and a second electrode that are stacked;
a light-emitting layer between the first electrode and the second electrode;
an electron transport layer between the first electrode and the light-emitting layer;
a hole blocking layer between the light-emitting layer and the electron transport layer, wherein
a material of the hole blocking layer includes one or more of compounds, containing coronene or cyclododecane, shown in the following formula (1)a and formula (1)b: 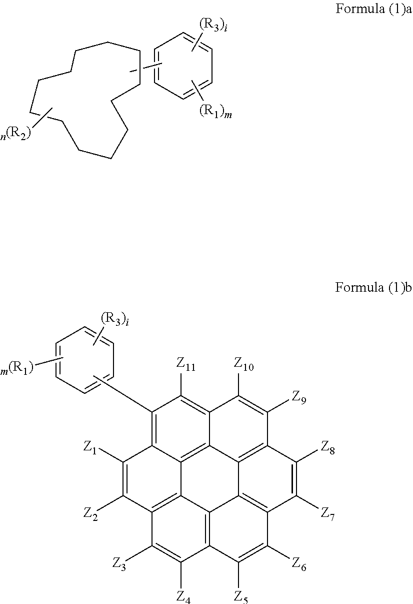
m is an integer from 0 to 2, n is an integer from 0 to 5, and i is an integer from 0 to 3; in the formula (1)a, at least one of m, n, and i is not 0; in the formula (1)b, at least one of m and i is not 0;
Z₁ to Z₁₁ are the same or different, and are each selected independently from any one of hydrogen (H) and a substituent
the substituent R, a substituent R₁ and a substituent R₂ are the same or different, and are each selected independently from any one of deuterium, halogen, cyano, nitryl, amino, C₁-C₄₀ alkyl, C₂-C₄₀ alkenyl, C₂-C₄₀ alkynyl, C₃-C₄₀ cycloalkyl, C₃-C₄₀ heterocycloalkyl, C₆-C₆₀ aryl, C₅-C₆₀ heteroaryl, C₁-C₄₀ alkoxy, C₆-C₆₀ aryloxy, C₃-C₄₀ alkylsilyl, C₆-C₆₀ arylsilyl, C₁-C₄₀ alkylboron, C₆-C₆₀ arylboron, C₆-C₆₀ arylphosphinylene, C₆-C₆₀ monoaryl or diaryl phosphino and C₆-C₆₀ arylamino, or are each independently combined with an adjacent group to form a condensed ring; and
R₃ is selected from any one of substituted or unsubstituted heterocyclyl and fused heterocyclyl.

2. The light-emitting device according to claim 1, wherein
i is not 0, and R₃ is selected from any one of following formulas (3)a, (3)b, (3)c, (3)d and (3)e; 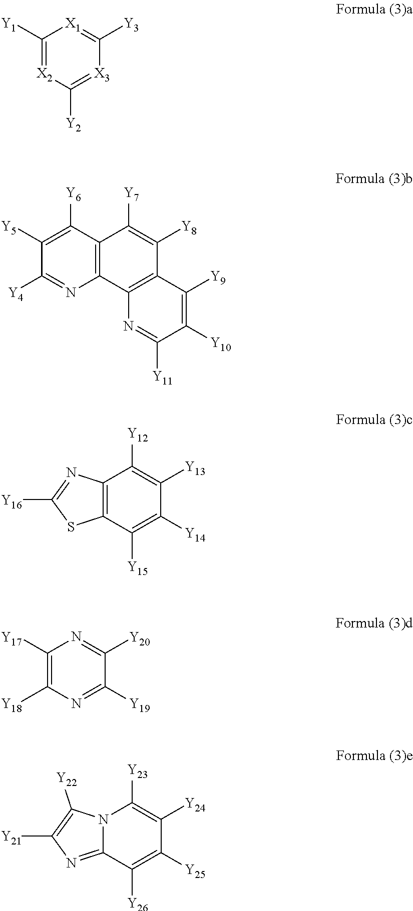
in the formula (3)a, X₁ to X₃ are each C(Y) or N, and at least two of X₁ to X₃ are N, wherein one of Y and Y₁ to Y₃ is combined with a dotted line in the formula (1)a or a dotted line in the formula (1)b, and others are the same or different, and are each selected independently from any one of the hydrogen and the substituent R;
in the formula (3)b, one of Y₄ to Y₁₁ is combined with the dotted line in the formula (1)a or the dotted line in the formula (1)b, and others are the same or different, and are each selected independently from any one of the hydrogen and the substituent R;
in the formula (3)c, one of Y₁₂ to Y₁₆ is combined with the dotted line in the formula (1)a or the dotted line in the formula (1)b, and others are the same or different, and are each selected independently from any one of the hydrogen and the substituent
in the formula (3)d, one of Y₁₇ to Y₂₀ is combined with the dotted line in the formula (1)a or the dotted line in the formula (1)b, and others are the same or different, and are each selected independently from any one of the hydrogen and the substituent R; and
in the formula (3)e, one of Y₂₁ to Y₂₆ is combined with the dotted line in the formula (1)a or the dotted line in the formula (1)b, and others are the same or different, and are each selected independently from any one of the hydrogen and the substituent R.

3. The light-emitting device according to claim 1, wherein
in the formula (1)a, at least m of m and n is not 0, and the substituent R₁ is selected from the structure shown in the following formula (2); 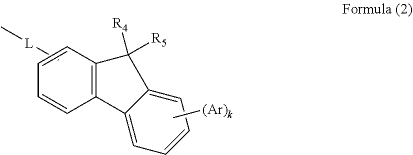
in the formula (2), R₄ and R₅ are the same or different, and are each selected independently from any one of deuterium, halogen, cyano, nitryl, C₁-C₄₀ alkyl, C₂-C₄₀ alkenyl, C₂-C₄₀ alkynyl, C₃-C₄₀ cycloalkyl, C₃-C₄₀ heterocycloalkyl, C₆-C₆₀ aryl, C₅-C₆₀ heteroaryl, C₁-C₄₀ alkoxy, C₆-C₆₀ aryloxy, C₃-C₄₀ alkylsilyl, C₆-C₆₀ arylsilyl, C₁-C₄₀ alkylboron, C₆-C₆₀ arylboron, C₆-C₆₀ arylphosphinylene, C₆-C₆₀ monoaryl or diaryl phosphino and C₆-C₆₀ arylamino, or are each independently combined with an adjacent group to form a condensed ring, wherein
k is an integer from 0 to 2, Ar is selected from any one of C₁-C₁₀ alkyl, C₃-C₁₀ cycloalkyl, and C₆-C₃₀ aryl, and L is selected from any one of a single bond, substituted or unsubstituted divalentaryl, and substituted or unsubstituted divalentheteroaryl.

4. The light-emitting device according to claim 1, wherein
m and n are both not 0, and the substituent R₂ and the substituent R₁ are the same or different.

5. The light-emitting device according to claim 4, wherein
n is greater than 1, and substituents R₂ are the same or different.

6. The light-emitting device according to claim 1, wherein
the material of the hole blocking layer includes one or more of the structures shown in the following formulas:

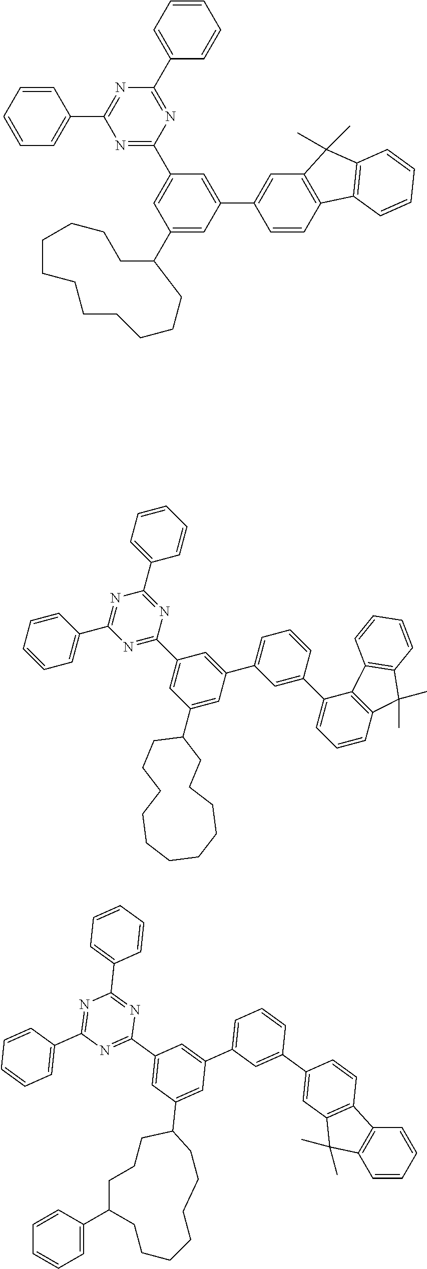 [SMILES: CC1(C)c2ccccc2-c2ccc(-c3cc(-c4nc(-c5ccccc5)nc(-c5ccccc5)n4)cc(C4CCCCCCCCCCC4)c3)cc21 ; CC1(C)c2ccccc2-c2c(-c3cccc(-c4cc(-c5nc(-c6ccccc6)nc(-c6ccccc6)n5)cc(C5CCCCCCCCCCC5)c4)c3)cccc21 ; CC1(C)c2ccccc2-c2ccc(-c3cccc(-c4cc(-c5nc(-c6ccccc6)nc(-c6ccccc6)n5)cc(C5CCCCCCCC(c6ccccc6)CCC5)c4)c3)cc21]

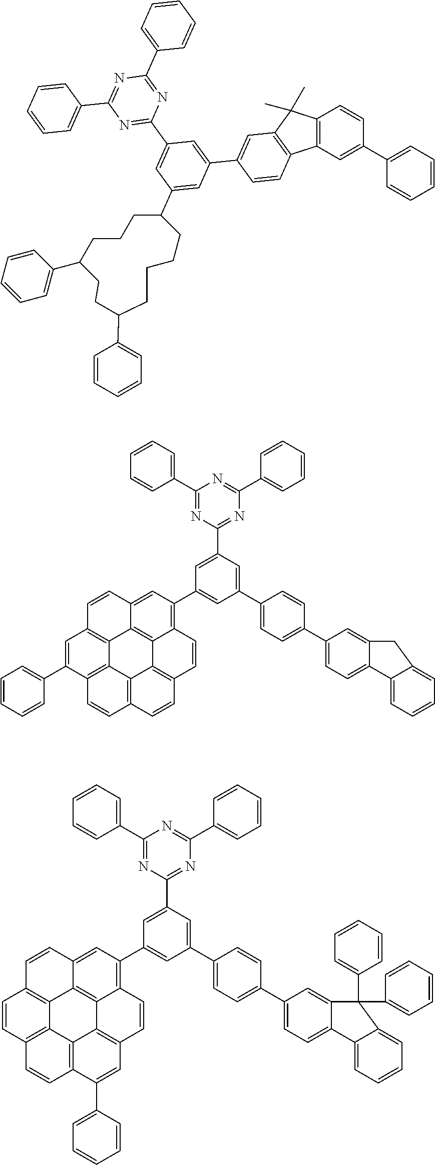 [SMILES: CC1(C)c2ccc(-c3ccccc3)cc2-c2ccc(-c3cc(-c4nc(-c5ccccc5)nc(-c5ccccc5)n4)cc(C4CCCCC(c5ccccc5)CCC(c5ccccc5)CCC4)c3)cc21 ; c1ccc(-c2nc(-c3ccccc3)nc(-c3cc(-c4ccc(-c5ccc6c(c5)Cc5ccccc5-6)cc4)cc(-c4cc5ccc6cc(-c7ccccc7)c7ccc8ccc9ccc4c4c9c8c7c6c54)c3)n2)cc1 ; c1ccc(-c2nc(-c3ccccc3)nc(-c3cc(-c4ccc(-c5ccc6c(c5)C(c5ccccc5)(c5ccccc5)c5ccccc5-6)cc4)cc(-c4cc5ccc6ccc7ccc8c(-c9ccccc9)cc9ccc4c4c5c6c7c8c94)c3)n2)cc1]

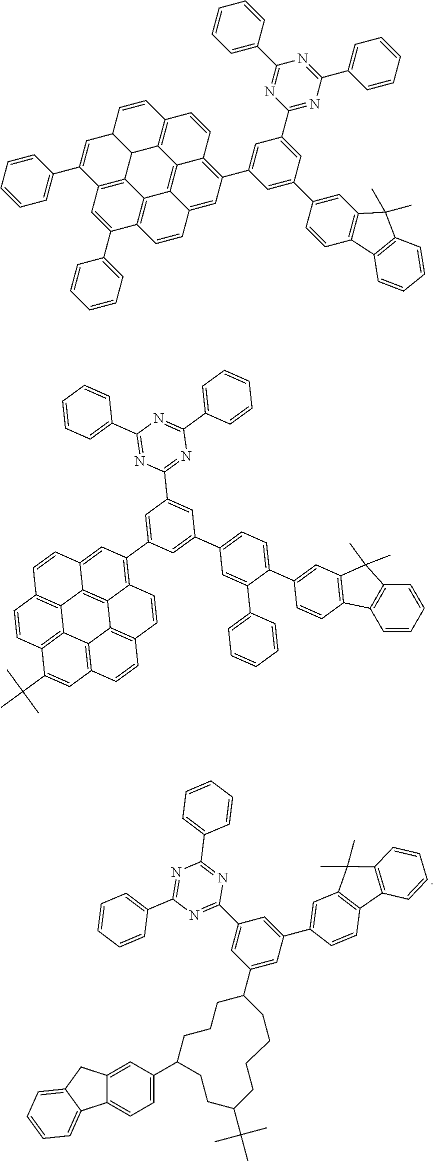 [SMILES: CC1(C)c2ccccc2-c2ccc(-c3cc(-c4nc(-c5ccccc5)nc(-c5ccccc5)n4)cc(-c4cc5ccc6c(-c7ccccc7)cc7c8c6c5c5c6c(ccc45)C=CC(C=C7c4ccccc4)C68)c3)cc21 ; CC(C)(C)c1cc2ccc3ccc4c(-c5cc(-c6ccc(-c7ccc8c(c7)C(C)(C)c7ccccc7-8)c(-c7ccccc7)c6)cc(-c6nc(-c7ccccc7)nc(-c7ccccc7)n6)c5)cc5ccc6ccc1c1c6c5c4c3c21 ; CC1(C)c2ccccc2-c2ccc(-c3cc(-c4nc(-c5ccccc5)nc(-c5ccccc5)n4)cc(C4CCCCC(C(C)(C)C)CCC(c5ccc6c(c5)Cc5ccccc5-6)CCC4)c3)cc21]

7. The light-emitting device according to claim 1, wherein
highest occupied molecular orbital (HOMO) energy levels of the compounds containing the coronene or the cyclododecane are less than -5.6 eV.

8. The light-emitting device according to claim 1, wherein
a highest occupied molecular orbital (HOMO) energy level of a compound in the compounds containing the coronene or the cyclododecane and a lowest unoccupied molecular orbital (LUMO) energy level of the compound containing the coronene or the cyclododecane meet the following formula:

|E_{HOMO}−E_{LUMO}|≥3.2 eV.

9. The light-emitting device according to claim 1, wherein
lowest singlet energy of the compounds containing the coronene or the cyclododecane is greater than 3.1 eV.

10. The light-emitting device according to claim 1, wherein
a difference between lowest singlet energy of a compound in the compounds containing the coronene or the cyclododecane and lowest triplet energy of the compound containing the coronene or the cyclododecane is greater than 0.51 eV.

11. The light-emitting device according to any one of claim 1, wherein
glass transition temperatures of the compounds containing the coronene or the cyclododecane are within a range of 136° C. to 153° C., inclusive.

12. The light-emitting device according to claim 1, wherein
an energy level difference between a highest occupied molecular orbital (HOMO) energy level of the light-emitting layer and a HOMO energy level of the hole blocking layer is greater than 0.2 eV.

13. The light-emitting device according to claim 1, wherein
an absolute value of an energy level difference between a lowest unoccupied molecular orbital (LUMO) energy level of the light-emitting layer and a LUMO energy level of the hole blocking layer is less than 0.3 eV.

14. The light-emitting device according to any one of claim 1, wherein
an absolute value of an energy level difference between a lowest unoccupied molecular orbital (LUMO) energy level of the hole blocking layer and a LUMO energy level of the electron transport layer is less than 0.3 eV.

15. The light-emitting device according to claim 1, wherein
an absolute value of an energy level difference between a highest occupied molecular orbital (HOMO) energy level of the hole blocking layer and a HOMO energy level of the electron transport layer is greater than 0.2 eV.

16. The light-emitting device according to any one of claim 1, wherein
a material of the electron transport layer includes one or more of the compounds, containing the coronene or the cyclododecane, shown in the formula (1)a and the formula (1)b.

17. The light-emitting device according to claim 1, wherein
a difference between energy of the hole blocking layer in a lowest singlet excited state and energy of the light-emitting layer in the lowest singlet excited state is greater than 0.2 eV, and a difference between energy of the hole blocking layer in a lowest triplet excited state and energy of the light-emitting layer in the lowest triplet excited state is greater than 0.2 eV.

18. The light-emitting device according to claim 1, wherein
a material of the light-emitting layer includes a host material and a guest material, the host material is selected from any one of anthracene, benzanthracene, benzophenanthrene and/or pyrene compounds and derivatives of these compounds, and atropisomers of these compounds and atropisomers of the derivatives of these compounds, and the guest material is selected from arylamino type compounds.

19. A light-emitting substrate, comprising the light-emitting device according to claim 1.

20. A light-emitting apparatus, comprising the light-emitting substrate according to claim 19.
